(19) United States
(12) Patent Application Publication
(10) Pub. No.: US 2025/0106461 A1
(43) Pub. Date: Mar. 27, 2025

(54) IMMUTABLE CHRONOLOGICAL ELECTRONIC RECORD SYSTEM

(71) Applicants: Yuh-shen SONG, Porter Ranch, CA (US); Catherine LEW, Porter Ranch, CA (US)

(72) Inventors: Yuh-shen SONG, Porter Ranch, CA (US); Catherine LEW, Porter Ranch, CA (US)

(21) Appl. No.: 18/763,925

(22) Filed: Jul. 3, 2024

Related U.S. Application Data
(60) Provisional application No. 63/584,839, filed on Sep. 22, 2023.

(51) Int. Cl. H04N 21/41; A61L 9/12; H04L 9/32

(52) U.S. Cl. CPC H04N 21/4131; A61L 9/12; H04L 9/3236

(57) ABSTRACT

A computerized method produces an immutable chronological electronic record system. The immutable chronological electronic record system uses the immutable electronic record technology to establish a multimedia art trading system, an electronic gift card issuance and management system, and other systems that need to maintain immutable chronological electronic records.

### CROSS-REFERENCE TO RELATED APPLICATION

The present application claims the benefit of U.S. Provisional Patent Application No. 63/584,839, filed on Sep. 22, 2023, and titled “IMMUTABLE CHRONOLOGICAL ELECTRONIC RECORD SYSTEM,” the disclosure of which is expressly incorporated by reference in its entirety.

### TECHNICAL FIELD

The present disclosure relates generally to immutable chronological electronic record technology. More specifically, the present disclosure relates to an electronic record system that maintains the originality and the accuracy of its records to support trading of electronic artwork, tangible and intangible goods or services, virtual currencies, rights, obligations, ownership, electronic financial instruments, etc., and other applications that require electronic records to be immutable and auditable.

### BACKGROUND

The history of Blockchain dates back to 2008 when an individual or group of individuals using the pseudonym “Satoshi Nakamoto” published a whitepaper titled “Bitcoin: A Peer-to-Peer Electronic Cash System.” The whitepaper outlined the concept of a decentralized digital currency (Bitcoin) and the underlying technology called Blockchain.

Although Blockchain is commonly used as the core technology for various types of cryptocurrencies including Bitcoin, Blockchain has numerous known weaknesses that make Blockchain-based cryptocurrencies unsuitable for many daily commercial transactions. For example, it may take about ten minutes to confirm a transaction on Blockchain. If a person uses a Blockchain-based cryptocurrency to purchase an ice cream, the ice cream will become watery cream by the time the transaction is confirmed.

In practice, for a Bitcoin transaction to be considered as fully secure, it is common to wait for at least six confirmations, which would typically take about one hour. This means that if the Bitcoin transaction involves an expensive trade, it takes about one hour for the transaction to be considered fully secure. Moreover, the value of cryptocurrency is so volatile that an originally profitable transaction based on a cryptocurrency may become a money-losing transaction by the time the deal is closed because the cryptocurrency has depreciated in value.

In addition to the above two well-known problems, there are other issues with Blockchain, such as scalability, energy consumption, storage requirements, governance challenges, lack of a regulatory framework, human error and smart contract vulnerabilities, immutability concerns, privacy and confidentiality issues, and adoption barriers.

Scalability: Blockchain faces scalability challenges as the size of the network grows. The decentralized nature of Blockchain requires all nodes to store and process every transaction, leading to potential bottlenecks and reduced transaction speeds. This issue becomes more pronounced in Blockchains with a large number of users.

Energy consumption: Blockchain mining, particularly in proof-of-work (PoW) consensus algorithms, demands substantial computational power and energy consumption. The energy-intensive nature of Blockchain operations has raised concerns about its environmental impact and sustainability.

Storage requirements: Each node in a Blockchain network stores a complete copy of the entire Blockchain, leading to significant storage requirements. As the Blockchain grows in size over time, it becomes increasingly challenging for participants with limited storage capacity to join the network.

Governance challenges: Blockchain governance refers to decision-making processes for protocol updates and network changes. Achieving consensus among participants with diverse interests can be complex and slow, leading to delays in implementing necessary updates or addressing security vulnerabilities.

Lack of regulatory framework: The decentralized and borderless nature of Blockchain technology poses challenges for regulators. The absence of a unified regulatory framework can hinder widespread adoption and create legal uncertainties in various areas such as data privacy, taxation, and intellectual property rights.

Human error and smart contract vulnerabilities: Smart contracts, which are self-executing contracts on the Blockchain, are susceptible to programming errors and vulnerabilities. If not written or audited properly, smart contracts can be exploited, leading to financial losses or unintended consequences.

Immutability concerns: While the immutability of Blockchain transactions is a strength for security purposes, it can also be a weakness in certain scenarios. If a fraudulent or erroneous transaction is recorded on the Blockchain, it becomes difficult to rectify or reverse the transaction without compromising the integrity of the entire chain.

Privacy and confidentiality: Blockchains, by design, are transparent, allowing anyone on the network to view the entire transaction history. While this transparency enhances trust and security, the transparency can be problematic for use cases that require privacy and confidentiality, such as sensitive business transactions or personal data storage.

Adoption barriers: Blockchain technology still faces challenges in terms of usability, user experience, and adoption barriers for non-technical users. The complexity of the underlying technology can limit mainstream acceptance and hinder widespread adoption.

Moreover, any money exchangers in the USA, including the Blockchain operators, need to comply with the Bank Secrecy Act, which was first established in 1970. Under the Bank Secrecy Act, money exchangers must report suspicious activities to the government. Suspicious activities cover a very broad scope of activities. For example, money laundering, terrorist financing, fraud, embezzlement, identity theft, computer intrusion, self-dealing, bribery, false statement, counterfeit instruments, mysterious disappearance, etc., are all classified as suspicious activities.

After the 9/11 tragedy, U.S. lawmakers believed that effective compliance with the Bank Secrecy Act could have prevented the 9/11 tragedy. To further enforce the Bank Secrecy Act, the U.S. Congress passed the USA PATRIOT Act, which enacted severe civil and/or criminal penalties for violations of the Bank Secrecy Act.

Furthermore, U.S. government agencies, such as Financial Crimes Enforcement Network (FinCEN), Office of Comptroller of Currency (OCC), Federal Reserve Bank (FRB), Federal Deposit Insurance Company (FDIC), National Credit Unions Administration (NCUA), State Banking Departments, Department of Financial Institutions, etc., strictly require financial institutions, including money exchangers, to comply with the Bank Secrecy Act, especially in their obligations to file Suspicious Activities Reports (SARs) to FinCEN.

The challenges noted for complying with the USA PATRIOT Act and the Bank Secrecy Act (BSA) are just some examples to illustrate the importance of identifying suspicious activities. Identifying suspicious activities can also be used to comply with other laws, such as the Fair and Accurate Credit Transactions Act (FACT Act), the Unlawful Internet Gambling Enforcement Act (UIGEA), the Elder Abuse Reporting Act, (EARA), the Sarbanes-Oxley Act (SOX), the regulations set by the Office of Foreign Assets Control (OFAC), and other laws and regulations.

A computerized system and network to resolve the weaknesses of Blockchain would be desirable.

### SUMMARY

The present disclosure includes a number of aspects that can be combined together to form a variety of computer systems and methods.

In some aspects of the present disclosure, a computerized method to produce a continuous, infinite, immutable, auditable, chronological electronic record uses a hash function to convert a first set of data stored in a database into a first hash value, the first set of data being a first module. The method also stores the first module and the first hash value into a read-only memory; and inserts the first hash value into a second set of data recorded in the database after the first set of data is recorded. The method uses the hash function to convert the second set of data stored in the database into a second hash value, the second set of data being a second module. The method also stores the second module and the second hash value into the read-only memory.

In this method, the hash function comprises at least one of SHA1, SHA2, SHA3, SHA256, and SHA512, another hash function, or a combination thereof. The first set of data comprises at least an electronic record related to a subject, which comprises a legal position related to at least one of a house, a building, a land, a mine, a car, a boat, an airplane, a company, an organization, a club, a security, a loan, a physical property, an intellectual property, an artwork, a virtual currency, a cryptocurrency, a financial instrument, another tangible property, another intangible property, or a combination thereof.

In general, a legal position comprises at least one of an ownership, a creditor, a debtor, a successor, a guarantor, a guarantee, a renter, a borrower, a lender, a licensee, a licensor, an assignee, an assignor, a trustee, a contractor, an employer, an employee, a buyer, a seller, an agent, a consultant, an advisor, an auditor, a singer, a custodian, a guardian, a defender, a caretaker, a guard, an attorney, a prosecutor, a district attorney, an accountant, a bookkeeper, an expert, a witness, a juror, a criminal, a judge, a plaintiff, an accuser, a complainant, a litigant, a claimant, an applicant, a defendant, a perpetrator, an offender, a respondent, a suspect, another type of position recognized by law, or a combination thereof.

In general, an electronic record comprises at least one of a transactional record, a non-transactional record, another type of record, or a combination thereof. The transactional record comprises at least one of a year, month, day, and time of the transaction; a dollar figure of the transaction; a purchase agreement identification number; a purchase agreement hash value; a video file identification number; a video file hash value; a scent control file identification number; a scent control file hash value; a member identification number of a buyer; a member identification number of a seller; and other information related to the transaction, or a combination thereof.

In general, a read-only memory comprises at least one of Compact Disc Read-Only Memory (CD-ROM), Digital Versatile Disc Read-Only Memory (DVD-ROM), backup computer system, Blu-ray Disc Read-Only Memory (BD-ROM), Masked ROM (MROM), Programmable Read-Only Memory (PROM), Erasable Programmable Read-Only Memory (EPROM), Electrically Erasable Programmable Read-Only Memory (EEPROM), Flash Memory, another type of read-only memory, or a combination thereof.

In general, a computer system comprises a device interface, which comprises at least one of a keyboard, a keypad, a monitor, a display, a terminal, a computer, a control panel, a vehicle dashboard, a network interface, a machine interface, an electrical interface, an electronic interface, a magnetic interface, an electromagnetic interface including an electromagnetic wave interface, an optical interface, a light interface, an acoustic interface, a video interface, an audio interface, a contactless interface, a mobile phone interface, a smartphone interface, a smartbook interface, a tablet interface, another communication device interface, a Personal Digital Assistant (PDA) interface, a handheld device interface, a portable device interface, a wireless interface, a wired interface, or a combination thereof.

The new method offers an innovative approach to keep electronic records error-proof, theft-proof, hacking-proof, and trustworthy through a new technology that is faster and more effective than Blockchain. The new method provides solid protection against error, modification, theft, fraud, and hacking.

In some aspects of the present disclosure, the computerized method further comprises inserting an Nth hash value converted from an Nth set of data into an (N+1)th set of data recorded in the database after the Nth set of data is recorded. The Nth set of data is the Nth module, where N is a natural number larger than 1. In these aspects, the hash function converts the (N+1)th set of data stored in the database into an (N+1)th hash value, the (N+1)th set of data being the (N+1)th module. These aspects also store the (N+1)th module and the (N+1)th hash value into the read-only memory.

In some aspects of the present disclosure, the computerized method further comprises repeating the above steps to continuously produce a series of modules.

In some aspects of the present disclosure, the computerized method further comprises retrieving an Nth hash value from the read-only memory, where N is a natural number. The method also retrieves an Nth hash value from a database; and confirms the data of the Nth module is intact when the Nth hash value from the read-only memory matches the Nth hash value from the database.

In some aspects of the present disclosure, the computerized method further comprises retrieving an Nth hash value from the read-only memory, where N is a natural number. The method also retrieves data of the Nth module from a database. The method uses the hash function to convert the data of the Nth module from the database into an Nth verification hash value. The method further confirms the data of the Nth module is intact when the Nth hash value from the read-only memory matches the Nth verification hash value.

In some aspects of the present disclosure, a computerized method to present a multimedia artwork comprises receiving a video file from a second computer system. The method also includes receiving a scent control file from the second computer system; sending to a third computer system the video file; and sending to the third computer system the scent control file. The method further includes instructing the third computer system to send the scent control file to a fourth computer system; instructing the third computer system to run the video file; and instructing the third computer system to synchronize the fourth computer to run the scent control file when the third computer system is running the video file.

In other aspects of the present disclosure, a multimedia artwork presentation apparatus comprises a scent releasing module configured to release at least one fragrance from one scent container with strength and timing specified by an electronic signal. The apparatus also includes a communication module configured to receive a scent control file and a schedule to run the scent control file. The apparatus further includes a control module configured to instruct the scent releasing module to select a fragrance from a scent container and release the selected fragrance at a strength and a time based on the scent control file received by the communication module.

In still other aspects of the present disclosure, a computerized method to produce an immutable and auditable electronic record comprises receiving at a first computer system from a second computer system a request for an action on a data element. The method also includes conducting the requested action on the data element by the first computer system, and using a hash function to produce a hash value by the first computer system based on a set of data that contains a result of the requested action on the data element. The method further includes sending from the first computer system to a third computer system the hash value and the set of data while the third computer system is used as a read-only memory device.

In some aspects of the present disclosure, a computerized method to audit data integrity of a second set of data comprises using a hash function to produce a first hash value from the first set of data and sending the first hash value to a second computer system. The method further comprises using the hash function to produce a second hash value from a second set of data where the first set of data is a historical record of the second set of data. Additionally, the method comprises receiving the first hash value from the second computer system and comparing the first hash value with the second hash value. As a result of the comparison, the method comprises confirming that the second set of data has not been altered since the first hash value was produced when the second hash value matches the first hash value. The second computer system in this method comprises at least a read-only memory.

In some aspects of the present disclosure, a computerized method to produce an immutable and auditable electronic record for a data field updated by a first computer system comprises running a second software program to produce a new set of data. The method further comprises using a hash function to produce a hash value from the new set of data and sending the hash value and the new set of data to a third computer system.

In this method, the second software program is produced by (1) searching a first software program running on the first computer system to identify an instruction that intends to write a data element into the data field, (2) modifying the first software program by inserting an instruction to write the data element into the new set of data, and (3) compiling the modified first software program to become the second software program. The first computer system and the second computer system can be integrated as one computer system. The third computer system comprises at least a read-only memory.

In some aspects of the present disclosure, a computerized method to maintain integrity of an electronic file comprises receiving the electronic file and creating a unique identification number for the electronic file. The method further comprises using a hash function to produce a hash value for the electronic file and saving the electronic file, the unique identification number, and the produced hash value together into a database. In addition, the method comprises saving at least the unique identification number and the produced hash value into a read-only memory.

In some aspects of the present disclosure, a computerized method to issue an electronic financial instrument comprises receiving a request to issue the electronic financial instrument and creating a unique identification number for the electronic financial instrument. The method further comprises retrieving an identification number associated with the second computer system and saving the unique identification number for the electronic financial instrument and the retrieved identification number associated with the second computer system into a database. Moreover, the method comprises using a hash function to produce a hash value from a set of data, which includes at least the unique identification number for the electronic financial instrument and the retrieved identification number associated with the second computer system; and saving the produced hash value and the set of data into a read-only memory.

For easy reference, this new technology is referred to as “Optimal Chain.” Optimal Chain provides a solution to detect suspicious activities so that the operators of Optimal Chains can easily report suspicious activities to the government organizations. Although virtual currency transactions, a multimedia art trading system, and an electronic gift card issuance and management system are used as examples in the present disclosure, Optimal Chain can be used for many other applications.

The above computerized methods of Optimal Chain, a multimedia artwork presentation apparatus, a multimedia art trading system, and an electronic gift card issuance and management system are merely examples. Many other methods and apparatuses can be formed by combining and rearranging the aspects of this disclosure.

This has outlined, rather broadly, the features and technical advantages of the present disclosure in order that the detailed description that follows may be better understood. Additional features and advantages of the disclosure will be described below. It should be appreciated by those skilled in the art that this disclosure may be readily utilized as a basis for modifying or designing other structures for carrying out the same purposes of the present disclosure. It should also be realized by those skilled in the art that such equivalent constructions do not depart from the teachings of the disclosure as set forth in the appended claims. The novel features, which are believed to be characteristic of the disclosure, both as to its organization and method of operation, together with further objects and advantages, will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

The features, nature, and advantages of the present disclosure will become more apparent from the detailed description set forth below when taken in conjunction with the drawings.

FIG. 1A illustrates a system and network diagram of Art Passion, a multimedia artwork trading system based on Optimal Chain, according to aspects of the present disclosure.

FIG. 1B illustrates a block diagram of a Scent Diffuser, according to aspects of the present disclosure.

FIG. 1C illustrates a chain structure of Optimal Chain, according to aspects of the present disclosure.

FIG. 1D illustrates a system and network diagram of Cheer Pay, an electronic gift card issuance and management system based on Optimal Chain, according to aspects of the present disclosure.

FIG. 1E illustrates the conversion of an existing software system to a new software system based on Optimal Chain, according to aspects of the present disclosure.

FIG. 1F illustrates a system and network diagram of an Intelligent Alert System according to aspects of the present disclosure.

FIGS. 2-8 are flowcharts for a multimedia artwork trading system according to aspects of the present disclosure.

FIGS. 9-11 are flowcharts for an electronic gift card issuance and management system according to aspects of the present disclosure.

The detailed description set forth below, in connection with the appended drawings, is intended as a description of various configurations and is not intended to represent the only configurations in which the concepts described may be practiced. The detailed description includes specific details for the purpose of providing a thorough understanding of the various concepts. However, it will be apparent to those skilled in the art that these concepts may be practiced without these specific details. In some instances, well-known structures and components are shown in block diagram form in order to avoid obscuring such concepts. As described, the use of the term “or” may mean either “inclusive OR” or “exclusive OR,” depending on the situation of the application based on the convention.

### DETAILED DESCRIPTION

Some aspects of the present disclosure are directed to a reliable, effective, efficient, continuous, infinite, immutable, auditable, chronological electronic record system which can be used to record and track electronic gift card issuance and management, virtual currency trading, electronic art trading, or trading of any tangible or intangible subjects which are represented or managed by electronic records. For example, these electronic records may include ownerships of houses, buildings, lands, mines, cars, boats, airplanes, companies, organizations, clubs, securities, loans, rights, obligations, physical properties, intellectual properties, electronic financial instruments, etc. Some aspects of the present disclosure are directed to a personal entertainment system, which empowers consumers to enjoy multimedia artworks. Some aspects of the present disclosure are directed to a regulatory compliance system, which empowers the operators of the electronic record system to comply with laws and regulations.

Bitcoin is the cryptocurrency on Blockchain. Bitcoin and Blockchain were introduced in 2008 when the global financial system was in turmoil. Some people thought that they could avoid the impact of the global financial crisis if they could bypass various government controls on the financial system. Bitcoin was created and a pseudonym approach was used so that the governments have no easy way to identify the owner of Bitcoin. To avoid fraud, Blockchain produces an immutable transactional record so that nobody can cheat by changing the record.

The inventors of Bitcoin assumed that a large number of computers on the Blockchain network can eliminate the need for any involvement by government. This assumption has flaws because greed is a common human trait. Like other human beings, computer engineers, such as hackers, can also use computer skills to commit crime. For example, the ownership of a Bitcoin is controlled by a private key. If the private key is stolen, the Bitcoin would be stolen as well. Because a pseudonym is used, a person who lost a Bitcoin cannot prove that he was the owner of the Bitcoin.

As a result, cryptocurrency has created a “lawless world” for criminals. Many crimes have occurred on Blockchains. For example, Bitcoin has been associated with various criminal activities due to its pseudonymous nature and potential for illicit transactions. Here are some well-known historical criminal events associated with Bitcoin.

Silk Road: Silk Road was an online marketplace operating from 2011 to 2013 that facilitated the sale of illegal goods and services, including drugs, fake passports, hacking tools, and more. Bitcoin was the primary currency used on Silk Road for anonymous transactions. The FBI shut down Silk Road in October 2013, and its founder, Ross Ulbricht, was arrested and later sentenced to life in prison.

Money laundering: Bitcoin's pseudonymous nature has made it attractive to individuals involved in money laundering schemes. Criminals have used Bitcoin to convert illicit funds into a seemingly legitimate form, taking advantage of its decentralized and borderless characteristics.

Ransomware attacks: Bitcoin has been the preferred payment method for ransomware attacks, where cybercriminals encrypt victims' data and demand a ransom payment in Bitcoin to unlock it. Notable examples include the WannaCry and NotPetya attacks in 2017.

Darknet markets: Bitcoin has been used extensively in various darknet markets, which are online platforms that facilitate the sale of illegal goods and services, including drugs, weapons, stolen data, and hacking tools.

Ponzi schemes and investment fraud: Bitcoin's popularity and the potential for high returns have attracted scammers who operate Ponzi schemes or investment frauds. These scammers promise unrealistic returns on Bitcoin investments, luring unsuspecting individuals to invest their funds, only to disappear with the money later.

The price of Bitcoin is very volatile because the price of Bitcoin is driven by greed, which is an unpredictable and unstable part of human nature. Every time there is a crime that demands payment in Bitcoin, the Bitcoin price is pushed up by the crime.

The price of Bitcoin has dropped tremendously recently after criminals stopped using Bitcoin as the payment method because the US government has forced the Blockchain operators to recover those Bitcoins from the criminals.

Many consumers have lost a lot of money because of Bitcoin. Fifteen years after Bitcoin and Blockchain were introduced in 2008, it has been proven in the real world that Bitcoin and Blockchain have created more problems than before.

Ironically, although Bitcoin and Blockchain were created in 2008 to be independent from any government control, the US government and many governments worldwide have closely monitored and controlled Bitcoin and Blockchain today (in 2023). The original goal of “government-free” Bitcoin and Blockchain is lost today. The reality is that Blockchains are controlled by a group of computers while these computers are controlled by human beings. These human beings still need to obey the orders of their governments. The original imagination of a “government-free world controlled by computers” has a fundamental flaw.

Although Bitcoin and Blockchain have lost their original attraction, they have created some valuable application ideas. For example, it may be desirable for governments worldwide to issue their own fiat virtual currencies. However, it is not desirable for them to use Blockchain because Blockchain has many problems as explained above.

The present application discloses a new technology, Optimal Chain, which has solved the problems of Blockchain, but retains the important “immutability” nature of Blockchain. Optimal Chain is ideal for applications that need to be controlled by a government, a company, an organization, a person, etc.

Blockchain achieves immutability through a combination of cryptographic techniques and consensus algorithms. Immutability means that once data is recorded on a Blockchain, it is extremely difficult to alter or tamper with that data without detection. Here is an overview of how Blockchain achieves immutability.

Distributed ledger: Blockchain is a distributed ledger technology, which means that many copies of the Blockchain are maintained across a network of computers (nodes). Each node has a copy of the entire Blockchain, ensuring redundancy and decentralization.

Block structure and linking: Data on a Blockchain is organized into blocks, where each block contains a set of transactions or other information. Each block is linked to the previous block using a cryptographic hash called the “hash pointer.” The hash pointer includes the hash of the previous block, ensuring the integrity and immutability of the entire chain.

Consensus mechanism: Blockchain employs consensus mechanisms, such as Proof of Work (PoW) or Proof of Stake (POS), to reach agreement on the state of the Blockchain among the participating nodes. These mechanisms ensure that a majority of the network agrees on the validity of new transactions or blocks before they are added to the Blockchain. Consensus prevents malicious actors from tampering with Blockchain because they would need to control a majority of the network's computing power or stake.

Security through decentralization: The decentralized nature of Blockchain, with its distributed network of nodes, makes it more resilient to attacks. In order to tamper with data on the Blockchain, an attacker would need to compromise a majority of the nodes simultaneously, which is highly unlikely in a well-designed and sufficiently decentralized Blockchain network.

Transparency: Blockchain's transparency allows participants to verify the entire transaction history. Because the Blockchain is distributed and open to public scrutiny, any attempts to modify data would be immediately apparent, as the altered data would differ from the copies maintained by other participants.

Optimal Chain of the present disclosure has the advantages of Blockchain based on a different approach. For ease of explanation, a trading system of artworks, which are displayed on a computer, is used to explain the functions of Optimal Chain. This trading system is referred to as “Art Passion.”

Many artists cannot make sufficient money from the sales of their artworks. The inequity is worse today because modern technologies can be used to duplicate their original artworks at a very low cost. For example, an original painting kept by the museum may be worth $100 million while its duplicate may only cost $100.

It is very easy to duplicate artwork displayed on computers. Unfortunately, most people cannot tell the differences between the originals and the duplicates. As a result, people hesitate to collect artwork displayed on computers. Consequently, it is very difficult for artists of these types of artworks to earn money to support their art creation activities.

For easy explanation purposes, we will refer to artwork displayed on computers as “multimedia artwork.”

According to aspects of the present disclosure, Art Passion creates a unique marketplace for artists and collectors to trade multimedia artwork on Art Passion. More importantly, Art Passion only allows the original artwork to be traded on Art Passion. To ensure the credibility of the originality, Art Passion uses the Optimal Chain technology to protect the originality of the multimedia artwork.

Bitcoin can become useful because Blockchain tracks each Bitcoin since it was created including all historical transactions. Blockchain can tell who (in term of pseudonym) owns a particular Bitcoin at a particular time. In other words, Blockchain can immediately confirm whether a person truly owns a Bitcoin.

Similarly, because Optimal Chain tracks each original multimedia artwork since it was created including all historical transactions, Optimal Chain can tell who owns a particular original multimedia artwork at a particular time. As a result, Optimal Chain can immediately tell whether a person truly owns the original multimedia artwork. Through Optimal Chain, Art Passion ensures that only the original multimedia artwork can be traded on Art Passion.

Due to the “uniqueness” nature of original multimedia artwork, their values will continue to increase when the artists become more famous. Art collectors can resell their collected multimedia artwork at higher prices in the marketplace provided by Art Passion to make profits.

After an artist has uploaded his original artwork to Art Passion, the artist sets an “asking price” for the original artwork. The artwork will be displayed under the “Gallery” of the artist on Art Passion. The Gallery is open to the public. All visitors, collectors, and artists can see the original artwork through the Internet. However, nobody is allowed to download any artwork from Art Passion. Although people can make screenshots (or screen copies) of the original artwork, such screenshots (or copies) are not valuable. Art Passion only allows original artwork to be uploaded to Art Passion. The originality of the artwork is preserved on Art Passion through Optimal Chain.

An art collector may place an offer to purchase the artwork. If the offer is higher than the asking price, the deal is closed and the art collector becomes the new owner of the artwork. After the art collector has purchased the multimedia artwork on Art Passion, the artwork will be displayed in the “Club” of the art collector on Art Passion. The art collector can decide whether to open the Club to the public, or keep it private. If the art collector decides to open the Club to the public, the art collector can charge a fee for visiting his Club. If the art collector decides to keep the Club private, only the art collector can enjoy the multimedia artwork.

Because the art collectors own the artwork kept in their Clubs, the art collectors have the option to move the artwork from their Clubs to their Galleries so that they can sell the artworks in the marketplace provided by Art Passion.

There are many ways to enjoy the multimedia artwork kept in the Clubs. For example, the visitors to the Clubs can “mirror” their computers, notebooks, mobile phones, etc., with any modern large screens so that they can enjoy the multimedia artworks at any place any time.

Art Passion provides a variety of methods to display multimedia artworks on the best displays with the best multimedia effects. In addition to displaying the video (which includes pictures and sounds) on the computer system of a viewer (e.g., consumer), Art Passion also sends a scent control file to the computer system of the consumer. The computer system of the consumer will send electronic signals to a scent diffuser near the consumer through Bluetooth or other modern data transmission methods. These electronic signals control the type of scent, the strength of the scent, and the timing of releasing the scent.

Alternatively, the computer system of the consumer can send the scent control file to the scent diffuser. The scent diffuser will control the type of scent, the strength of the scent, and the timing of releasing the scent based on the scent control file.

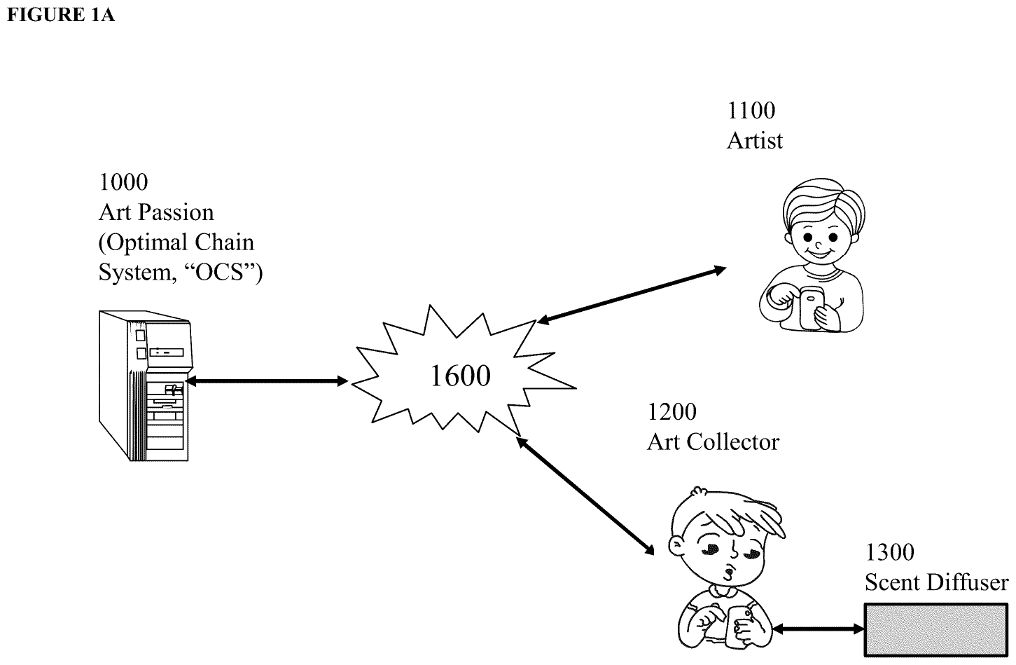

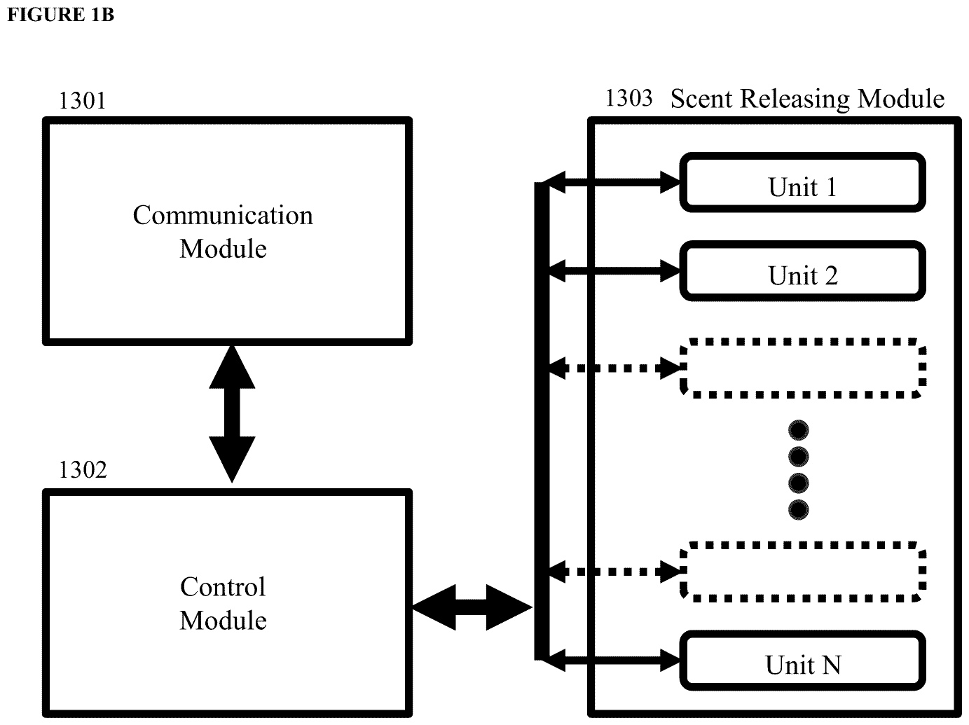

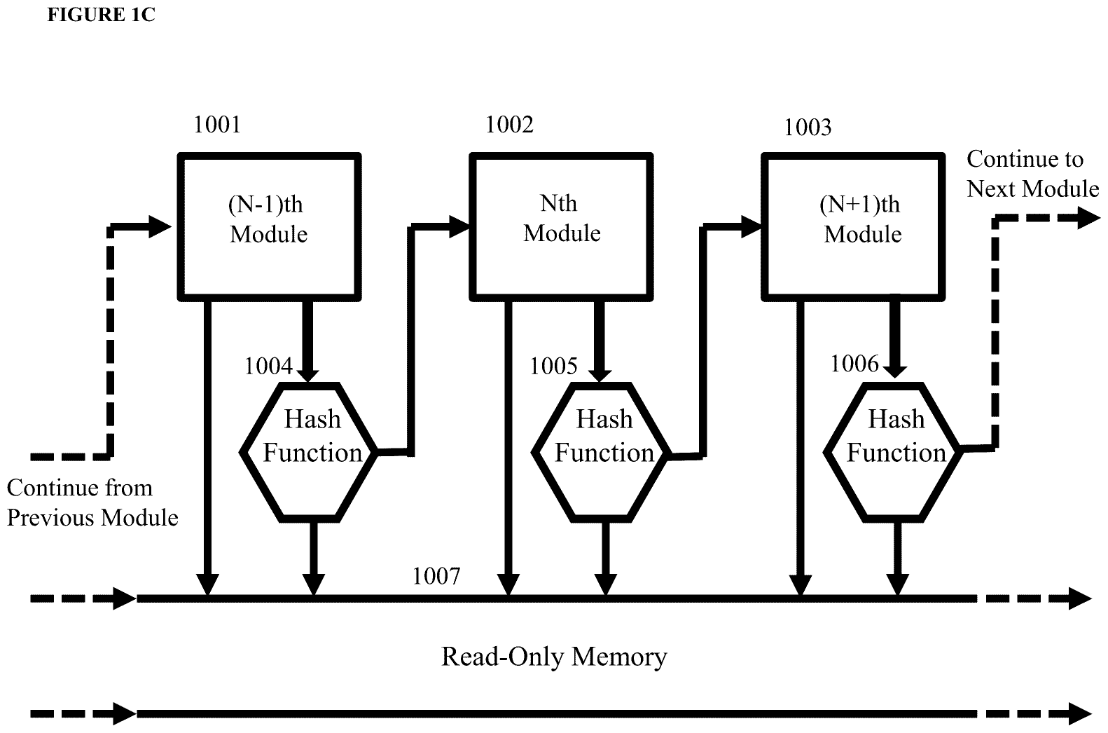

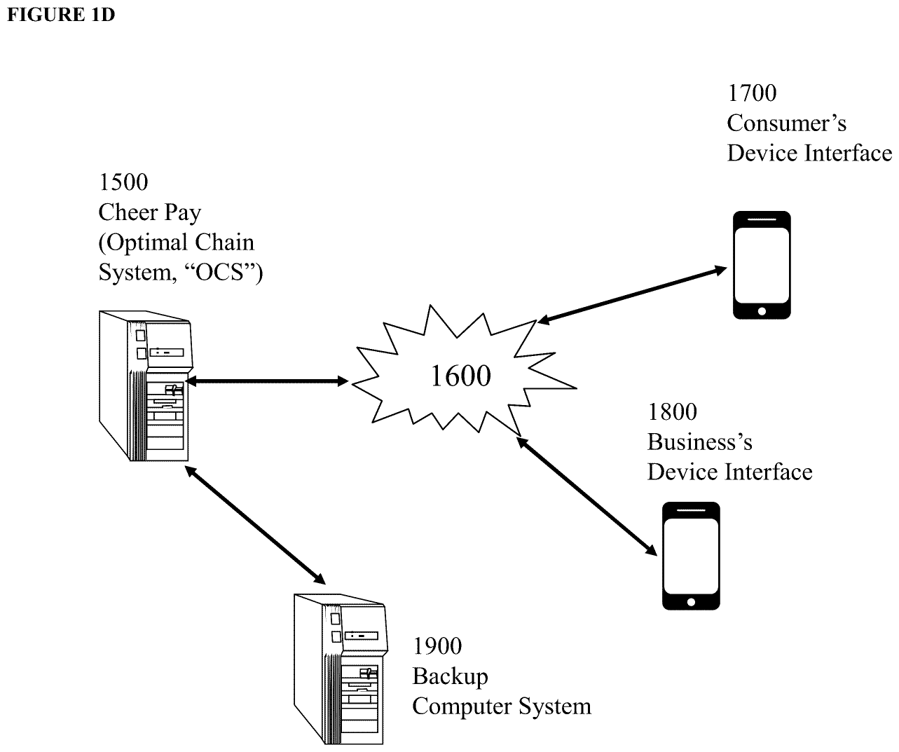

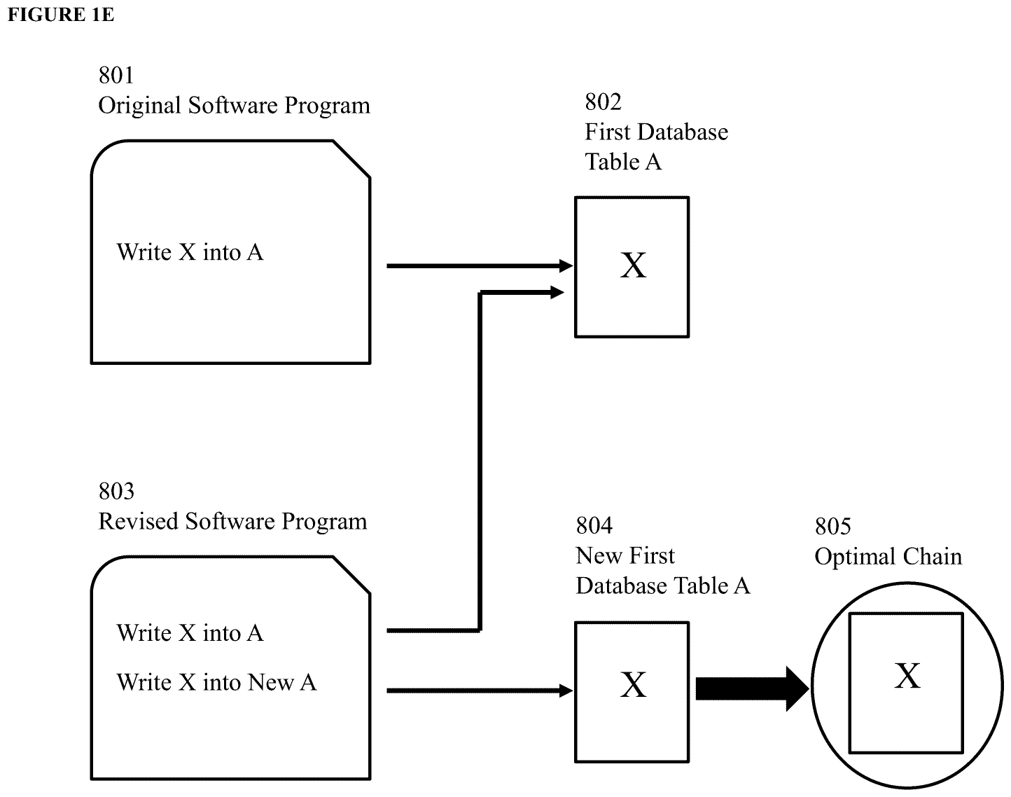

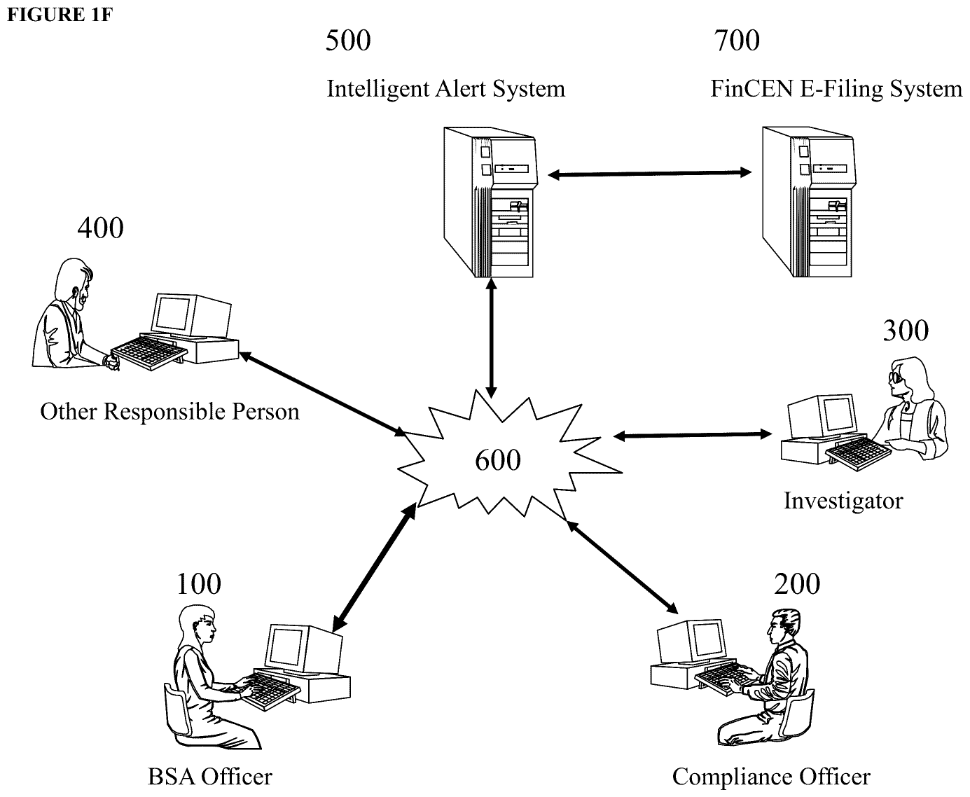

FIG. 1A illustrates an example of Art Passion 1000 (e.g., an Optimal Chain system) and a computer network 1600, such as the Internet, according to aspects of the present disclosure. In one configuration, Art Passion 1000 enables an artist 1100 to present a multimedia artwork to an art collector 1200.

Art Passion 1000 enables the artist 1100 of the original artwork to create the scent control file that enables the Scent Diffuser 1300 to release a certain type of scent with certain strength at a certain timing. For example, the artist can release a light ocean fragrance at the beginning of the video, then change to a strong rose fragrance at the 9th second of the video, then change to a strong gardenia fragrance at the 15th second of the video, then change to a medium cedar wood fragrance at the 21st second of the video, etc.

Art Passion 1000 will send the video file to the computer system (e.g., desktop, laptop, notebook, phone, etc.) of the art collector 1200 to display the video (e.g., pictures and sounds). Simultaneously, Art Passion 1000 will send the scent control file to the computer system of the art collector 1200. The computer system of the art collector 1200 sends the scent control file to a scent diffuser 1300 and synchronizes the timing with the video so that the scent diffuser 1300 releases different fragrances at the strength and timing set by the artist 1100 when the video is played. As a result, the art collector 1200 can enjoy the multimedia artwork created by the artist 1100 through a full scope of sensory perception (e.g., eyes, ears, and nose).

FIG. 1B illustrates an example of the scent diffuser 1300, which consists of three (3) modules. A communication module 1301 receives the scent control file from the computer system of the art collector 1200. Then, the communication module 1301 sends the scent control file to the control module 1302. The scent control file provides a schedule that releases different fragrances at different times with different strengths.

A scent releasing module 1303 consists of a number of units. Each unit has a container that stores the source material of a fragrance. For example, if Unit 1 releases a rose fragrance, the container stores rose perfume, rose oil, or similar materials that smell like roses. Additionally, Unit 1 has an electronic valve that is controlled by electronic signals sent from the control module 1302.

For example, when the electronic valve of Unit 1 is fully opened by the control module 1302, the rose fragrance is fully released from the container of Unit 1. When the electronic valve of Unit 1 is fully closed by the control module 1302, the rose fragrance is not released from the container of Unit 1. When the electronic valve of Unit 1 is partially opened by the control module 1302, the rose fragrance is partially released from the container of Unit 1. As a result, the control module 1302 can control the strength of the rose fragrance released from Unit 1.

Because different Units may contain different types of fragrances, according to the scent control file provided by the communication module 1301, the control module 1302 can instruct the scent releasing module 1303 to release a particular type of fragrance at a particular time with a particular strength. Moreover, the communication module 1301 receives an instruction from the computer system of the art collector 1200 to synchronize the running time of the scent control file so that the scent diffuser 1300 will precisely release the fragrances to match the video played on the computer system of the art collector 1200 based on the original design of the artist 1100. For example, the instruction to synchronize the running time of the scent control file can be “running the scent control file now,” or “running the scent control file at a particular time (such as 08:32:15 on Apr. 1, 2023).”

The scent diffuser 1300 is a computer system (e.g., a device interface). The communication module 1301, the control module 1302, and the scent releasing module 1303 can be implemented with at least one of a microprocessor, a RAM, an EPROM, other peripheral devices, or a combination thereof.

Art Passion is built on Optimal Chain, which is a continuous, infinite, immutable, auditable, chronological, electronic record system. Because Optimal Chain ensures that only the original artwork is traded on Art Passion, artists and art collectors can comfortably trade their artworks on Art Passion.

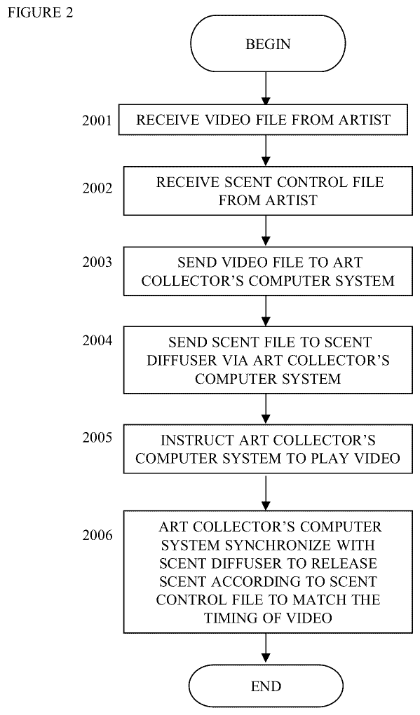

FIG. 2 illustrates an example of a flow chart for the artist 1100 to present the multimedia artwork to the art collector 1200. First, the artist 1100 creates a video file and a scent control file such that the release of the scent matches the play of the video to give the art collector 1200 a full scope of sensory pleasure.

In one configuration, as shown in FIG. 2 together with FIG. 1A, at block 2001, a central computer system (e.g., a device interface) 1000 receives the video file from the artist 1100 through the Internet 1600. At block 2002, the central computer system 1000 receives the scent control file from the artist 1100 through the Internet 1600. At block 2003, the central computer system 1000 sends the video file to the computer system of the art collector 1200 through the Internet 1600. At block 2004, the central computer system 1000 sends through the Internet 1600 the scent control file to the computer system of the art collector 1200, which then sends the scent control file to the scent diffuser 1300. At block 2005, the central computer system 1000 instructs the computer system of the art collector 1200 to show the video based on the received video file. At block 2006, the computer system of the art collector 1200 synchronizes with the scent diffuser 1300 to release scent according to the scent control file such that the scent diffuser 1300 releases the scent to match the timing of the video shown by the computer system of the art collector 1200 according to the type of scent and the strength of scent designed by the artist 1100.

The scent diffuser 1300 is a computer system (e.g., device interface) which has at least one microprocessor and one RAM device. In addition, the scent diffuser 1300 has at least one small scent container that contains the material (e.g., perfume, oil, etc.) that can produce the fragrance. The microprocessor of the scent diffuser 1300 controls the timing and the strength of releasing the fragrance. If there are multiple scent containers, the microprocessor of the scent diffuser 1300 controls each scent container separately such that different fragrances can be released at different timings with different strengths.

The microprocessor of the scent diffuser 1300 communicates with the computer system of the art collector 1200 via a wireless communication method, such as Bluetooth, Wi-Fi, or other similar communication methods.

The computer system of the art collector 1200 instructs the microprocessor of the scent diffuser 1300 to perform the tasks listed in the scent control file such that the microprocessor of the scent diffuser 1300 controls each scent container to release a certain strength of fragrance at a certain time. For example, according to the scent control file, the microprocessor of the scent diffuser 1300 can release a light ocean fragrance at the beginning of the video, then change to a strong rose fragrance at the 9th second of the video, then change to a strong gardenia fragrance at the 15th second of the video, then change to a medium cedar wood fragrance at the 21st second of the video, etc.

As a result, the art collector 1200 can enjoy the multimedia artwork through the video file and the scent control file created by the artist 1100 when the computer system of the art collector 1200 synchronizes with the microprocessor of the scent diffuser 1300 such that the computer system of the art collector 1200 plays the video according to the video file and, in parallel, the microprocessor of the scent diffuser 1300 releases the fragrances according to the scent control file. The computer system of the artist 1100, the central computer system 1000, the computer system of art collector 1200, and the computer system of the scent diffuser 1300 jointly form a computer system and network, e.g., Art Passion, to present the multimedia artwork created by the artist 1100 to the art collector 1200.

Hashing is a process of converting a set of data or a variable-length input into a fixed-size string of characters. The output generated by hashing is commonly referred to as a “hash” or “hash value.” The primary purpose of hashing is to create a unique representation of the input data, such that even a small change in the input will produce a significantly different hash value.

Hash functions are mathematical techniques used to perform the hashing process. These functions take an input and apply a series of complex calculations to produce a fixed-size output. The output is typically a sequence of characters or bits, often represented in hexadecimal or binary format.

Hash functions can be used to verify the integrity of data. For example, when the hash values of two sets of input data are different, one can instantly tell that these two sets of input data are different. Therefore, the hash functions can be used to instantly audit whether two sets of data are identical. For example, a screen copy of an original multimedia artwork and the original multimedia artwork are two different files. Consequently, their hash values are different. If a thief has stolen an original multimedia artwork and replaced it with a screen copy, such a crime can be instantly identified by a computer system (although human beings cannot see the differences) because the hash value of the copy is different from the hash value of the original multimedia artwork.

The computer system of Art Passion stores the hash value of the original multimedia artwork into a read-only memory, such as CD ROM, DVD ROM, PROM, backup computer system, Blu-ray Discs, etc. As a result, the computer system of Art Passion can easily audit whether a current multimedia artwork is the original multimedia artwork by producing the hash value of the current multimedia artwork and comparing the currently produced hash value with the hash value of the original multimedia artwork stored in the read-only memory. If these two hash values are different, the current multimedia artwork is not the original multimedia artwork.

SHA1, SHA2, SHA3, SHA256, and SHA512 are cryptographic hash functions that are part of the Secure Hash Algorithm (SHA) family, developed by the National Security Agency (NSA) in the United States. SHA1 (Secure Hash Algorithm 1) produces a 160-bit hash value, represented as a 40-digit hexadecimal number. However, SHA1 is considered to be insecure for many cryptographic applications due to vulnerabilities discovered over time. It is no longer recommended for use in new systems.

SHA2 (Secure Hash Algorithm 2) is an improved version of SHA1 and provides stronger security. SHA2 includes several variants that differ in the output size of the hash value. The most common variants are SHA256 and SHA512. SHA256 produces a 256-bit hash value, represented as a 64-digit hexadecimal number. SHA256 is widely used and considered secure for many cryptographic applications. SHA512 produces a 512-bit hash value, represented as a 128-digit hexadecimal number. SHA512 offers a higher level of security compared to SHA256, but requires more computational resources.

SHA3 (Secure Hash Algorithm 3) is the latest member of the SHA family. SHA3 was designed as a replacement for SHA2, addressing some potential weaknesses. SHA3 also offers various hash sizes, including 224, 256, 384, and 512 bits.

Optimal Chain uses the above hash functions or similar hash functions to perform various audit functions as explained below.

Each user of Art Passion opens an account to become a member of Art Passion. Art Passion assigns to each member a unique “member identification number,” which can be a long alpha-numerical number (e.g., 16 bytes). As a result, each artist and each art collector can be uniquely identified by the member identification number.

Art Passion uses modern database technologies to store and process data. When an original video file and an original scent control file are uploaded to Art Passion by an artist, Art Passion uses a hash function (e.g., SHA 256, SHA 512, other hash functions, etc.) to convert the video file into a “video file hash value;” and the scent control file into a “scent control file hash value.” These two hash values are stored in the database. They may be stored into the read-only memory of Optimal Chain.

In addition, Art Passion assigns to the original video file a unique “video file identification number,” which can be a long alpha-numerical sequence (e.g., 32 bytes). Art Passion also assigns to the original scent control file a unique “scent control file identification number,” which can be a long alpha-numerical sequence (e.g., 32 bytes). The video file identification number and the scent control file identification number are stored in the database associated with the video file hash value and the scent control file hash value.

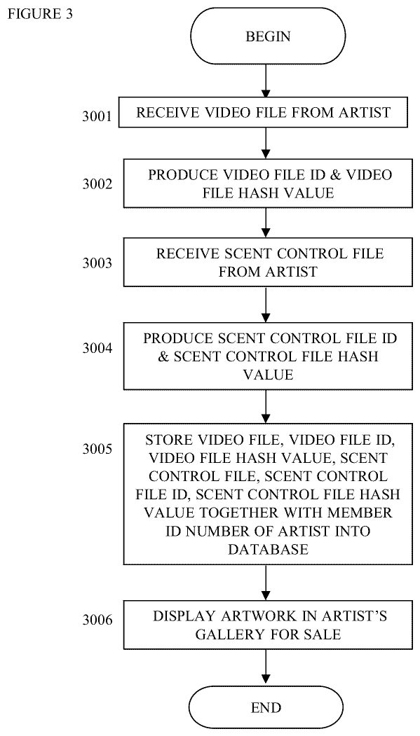

In one configuration, as shown in FIG. 3 together with FIG. 1A, at block 3001, the central computer system (e.g., a device interface) 1000 receives the video file from the Artist 1100 through the Internet 1600.

At block 3002, the central computer system 1000 produces a unique “video file identification number” for the video file. In addition, the central computer system 1000 uses a hash function to produce a “video file hash value” for the video file.

At block 3003, the central computer system 1000 receives the scent control file from the artist 1100 through the Internet 1600.

At block 3004, the central computer system 1000 produces a unique “scent control file identification number” for the scent control file. In addition, the central computer system 1000 uses a hash function to produce a “scent control file hash value” for the scent control file.

At block 3005, the central computer system 1000 stores into a database the member identification number of the artist 1100 together with the video file, the video file identification number, the video file hash value, the scent control file, the scent control file identification number, and the scent control file hash value.

The above process establishes a set of data that uniquely identifies the artist 1100 together with the video file and the scent control file created by the artist 1100. Additionally, this set of data may be stored into Optimal Chain so that nobody can modify this set of data. Because the data in Optimal Chain is immutable, this set of data in Optimal Chain may be used to quickly authenticate the video file and the scent control file. As an option to save storage space of Optimal Chain, the video file hash value is stored into Optimal Chain without the need to store the video file into Optimal Chain.

At block 3006, after the records have been established for the uploaded video file and scent control file, the central computer system 1000 instructs the computer system of the artist 1100 to display the artwork (e.g., pictures, sounds, and fragrances) of the artist 1100 in the gallery of the artist 1100 on Art Passion. The artwork is ready for sale on Art Passion.

To avoid unnecessary commercial disputes, Art Passion acts as an escrow agent between a buyer and a seller of an artwork. After the buyer has offered a price that is higher than the seller's asking price of the artwork, the deal enters an escrow process.

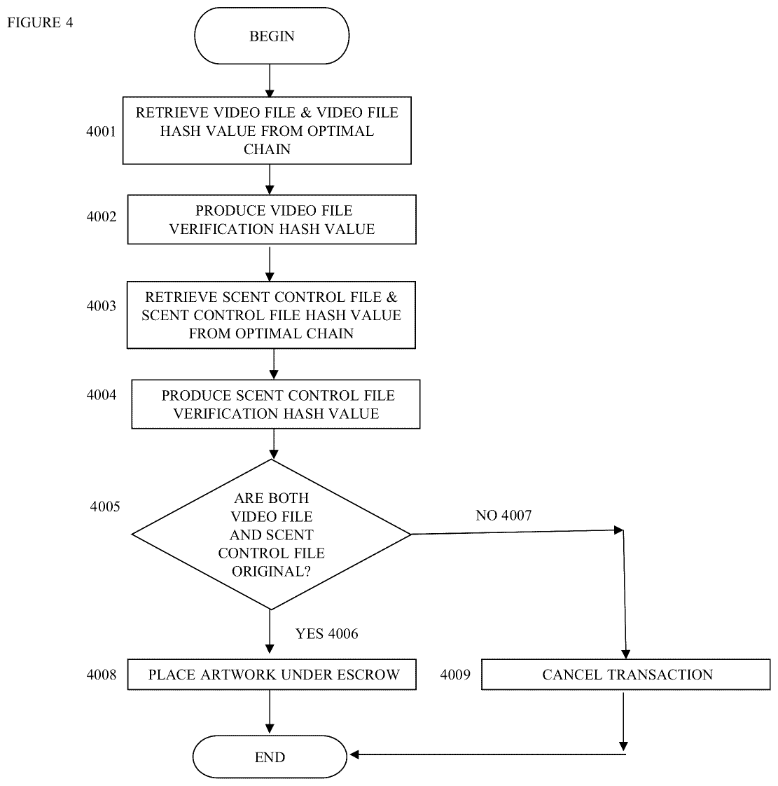

In one configuration, as shown in FIG. 4 together with FIG. 1A, at block 4001, the central computer system (e.g., a device interface) 1000 retrieves from Optimal Chain the video file of the artwork to be purchased and its associated video file hash value.

At block 4002, the central computer system 1000 applies the hash function to the retrieved video file to produce a new hash value, which is called the “video file verification hash value.” If the video file verification hash value matches the video file hash value retrieved from Optimal Chain, the retrieved video file is authenticated as the original video file uploaded by the artist 1100.

At block 4003, the central computer system (e.g., a device interface) 1000 retrieves from Optimal Chain the scent control file of the artwork to be purchased and its associated scent control file hash value.

At block 4004, the central computer system 1000 applies the hash function to the retrieved scent control file to produce a new hash value, which is called the “scent control file verification hash value.” If the scent control file verification hash value matches the scent control file hash value retrieved from Optimal Chain, the retrieved scent control file is authenticated as the original scent control file uploaded by Artist 1100.

At decision block 4005, by comparing the hash values as described above, the central computer system 1000 determines whether both the video file and the scent control file are the originals uploaded by the artist 1100.

If the answer is “yes” (branch 4006), the central computer system 1000 places the artwork, which includes the video file and the scent control file, into escrow (block 4008). If the answer is “no” (branch 4007), the central computer system 1000 cancels the purchase transaction (block 4009) because the artwork has failed to pass the authentication process described above.

If the artwork has passed the authentication process, the artwork is the original artwork uploaded by the artist 1100. The buyer and the seller move forward to close escrow based on the following process.

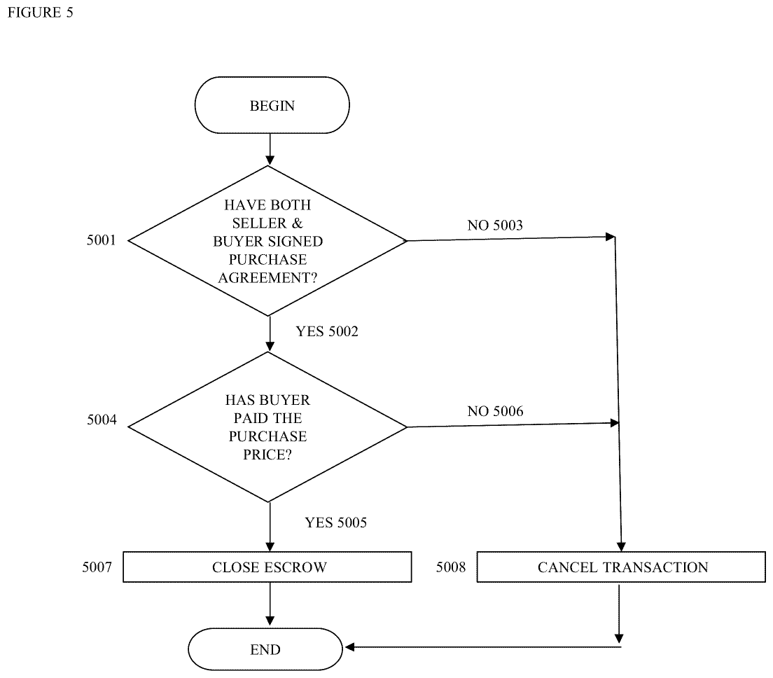

In one configuration, as shown in FIG. 5 together with FIG. 1A, at decision block 5001, the central computer system (e.g., a device interface) 1000 determines whether both the buyer and the seller have signed an irrevocable purchase agreement.

If the answer is “no” (branch 5003), the central computer system 1000 cancels the purchase transaction (block 5008). If the answer is “yes” (branch 5002), the central computer system 1000 determines whether the buyer has paid the fee based on the purchase price agreed upon by both the buyer and the seller (decision block 5004).

If the answer is “no” (branch 5006), the central computer system 1000 cancels the purchase transaction (block 5008).

If the answer is “yes” (branch 5005), the central computer system 1000 closes escrow. Once the escrow is closed, the central computer system 1000 records the buyer as the new owner of the artwork. Art Passion will move the artwork from the seller's Gallery to the buyer's Club on Art Passion.

The above escrow process ensures that neither the buyer nor the seller can cheat in the purchase transaction.

After the central computer system 1000 closes escrow, the central computer system 1000 (e.g., Art Passion) assigns to the purchase agreement a “purchase agreement identification number,” which can be a long alpha-numerical sequence (e.g., 32 bytes). Art Passion uses a hash function (e.g., SHA 256, SHA 512, other hash functions, etc.) to convert the purchase agreement file into a “purchase agreement hash value.” Art Passion stores into the database the purchase agreement identification number and the purchase agreement hash value.

In one configuration of the present disclosure, when there is a transaction in which a buyer acquires the artwork from a seller, the central computer system 1000 (e.g., Art Passion) records in the database the “transactional details” which include at least one of the year, month, day, and time of the transaction; the dollar figure of the transaction; the purchase agreement identification number; the purchase agreement hash value; the video file identification number; the video file hash value; the scent control file identification number; the scent control file hash value; the member identification number of the buyer; the member identification number of the seller of the artwork; and other information related to the transaction. The transaction details uniquely define the purchase transaction. The video file and the scent control file can also be included as a part of the transactional details.

In one configuration of the present disclosure, the central computer system 1000 (e.g., Art Passion) creates one “module” every second to establish Optimal Chain. The central computer system 1000 assigns to each module a “module identification number,” which can be a long alpha-numerical sequence (e.g., 48 bytes). The module contains (1) the module identification number; (2) year, month, day, and time of the creation of the module; (3) the transactional details of all transactions occurring after the creation of the previous module; (4) the module hash value of the previous module; and (5) other information related to the module.

Additionally, the central computer system 1000 (e.g., Art Passion) uses a hash function (e.g., SHA 256, SHA 512, etc.) to convert the data in the current module into a “module hash value” of the current module. This module hash value of the current module will be included in the contents of the next module that will be created at the next time unit (e.g., next second).

FIG. 1C illustrates an example of an Optimal Chain according to aspects of the present disclosure. In FIG. 1C, the symbol “Nth module” (block 1002) indicates one typical module in Optimal Chain at the nth position. The symbol “(N−1)th module” (block 1001) indicates the module before the Nth module. The symbol “(N+1)th module” (block 1003) indicates the module after the Nth module.

At block 1004, the central computer system 1000 converts the contents of “(N−1)th module” into an “(N−1)th module hash value.” The “(N−1)th module hash value” is included in the contents of the “Nth module” (block 1002). If the “(N−1)th module” is the first module of Optimal Chain, the “(N−1)th module” does not include the hash value of the previous module because the previous module does not exist. Alternatively, a special value (which indicates the module as the first module of Optimal Chain) can be used as the hash value for the previous module, which does not exist.

At block 1005, the central computer system 1000 converts the contents of “Nth module” into the “Nth module hash value.” The “Nth module hash value” is included in the contents of the “(N+1)th module” (block 1003).

As a result of the Optimal Chain structure shown in FIG. 1C, it is impossible for a hacker to modify any value in any specific module of Optimal Chain without modifying all modules after the specific module.

Moreover, the central computer system 1000 stores the “contents of the (N−1)th module” and the “(N−1)th module hash value” into a read-only memory (block 1007), which comprises, for example, Compact Disc Read-Only Memory (CD-ROM), Digital Versatile Disc Read-Only Memory (DVD-ROM), backup computer system, Blu-ray Disc Read-Only Memory (BD-ROM), Masked ROM (MROM), Programmable Read-Only Memory (PROM), Erasable Programmable Read-Only Memory (EPROM), Electrically Erasable Programmable Read-Only Memory (EEPROM), Flash Memory, or other read-only memory. These read-only memory devices ensure that the data inside each module is immutable.

As shown in FIG. 1C, the central computer system 1000 stores the “contents of the Nth module” and the “Nth module hash value” into read-only memory (block 1007). The central computer system 1000 also stores the “contents of the (N+1)th module” and the “(N+1)th module hash value” into read-only memory (block 1007).

FIG. 1C illustrates the relationships among any three consecutive modules of Optimal Chain. The contents of each module of Optimal Chain and the hash value of each module are saved into read-only memory (block 1007). As a result, no hacker can modify data of Optimal Chain.

By including the module hash value of the previous module into the contents of the current module, which will be used to create the module hash value of the current module that will be included as a part of the contents of the next module, all modules are linked sequentially together through these module hash values. Therefore, these modules have formed an auditable Optimal Chain.

The timing of producing one module per second is used for explanation purposes. A module can be created at any speed that is suitable for the application. For example, a module can be created faster at every 10 milli-seconds, or slower at every 15 seconds. The speed of module creation for Optimal Chain can be adjusted based on the needs of the application.

In comparison, Blockchain needs about 10 minutes to create one block. Optimal Chain is much faster than Blockchain.

In one application of the present disclosure, the records in the read-only memory are used to audit the records in the database, which is used to form the module for Optimal Chain.

An audit can be automatically performed by a computer system, which can be either a part of the central computer system 1000 or an independent computer system. For example, a simple audit on a specific module can be performed by comparing the module hash value of the specific module stored in the database with the module hash value of the same specific module stored in the read-only memory. If these two values are identical, the specific module has passed the simple audit.

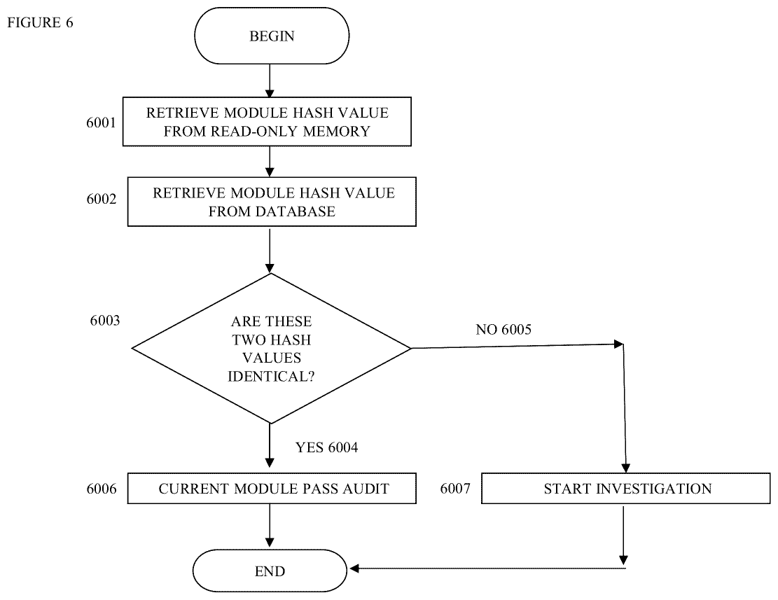

In one configuration, as shown in FIG. 6 together with FIG. 1C, at block 6001, the central computer system (e.g., a device interface) 1000 retrieves the module hash value of a specific module from the read-only memory.

At block 6002, the central computer system 1000 retrieves the module hash value of the same specific module from the database.

At decision block 6003, the central computer system 1000 determines whether these two module hash values are identical.

If the answer is “yes” (branch 6004), the specific module has passed the simple audit (block 6006).

If the answer is “no” (branch 6005), the specific module has failed the simple audit. Under such circumstances, Art Passion starts an investigation on this matter (block 6007).

For example, a comprehensive audit can be performed by comparing every data element between the specific module stored in the database and the same specific module stored in the read-only memory. If all the data elements of these two modules are identical, the specific module has passed the comprehensive audit. As explained earlier, the hash value of a set of data can represent the entire set of data in such a comparison.

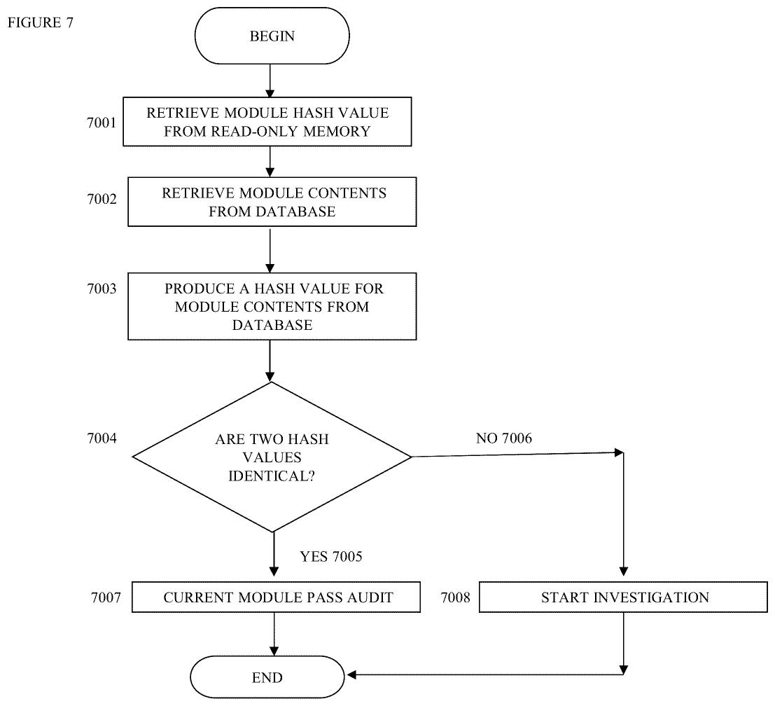

In one configuration, as shown in FIG. 7 together with FIG. 1C at block 7001, the central computer system (e.g., a device interface) 1000 retrieves the module hash value of a specific module from the read-only memory.

At block 7002, the central computer system 1000 retrieves the contents of the same specific module from the database.

At block 7003, the central computer system 1000 uses the hash function to convert the contents of the specific module retrieved from the database into a new hash value.

At decision block 7004, the central computer system 1000 determines whether the module hash value retrieved from the read-only memory and the hash value produced from the contents of the same specific module from database are identical.

If the answer is “no” (branch 7006), Art Passion starts an investigation on this matter (block 7008). If the answer is “yes” (branch 7005), the specific module has passed the audit process (block 7007).

A computer system can complete the above audit process of a specific module within a matter of milli-seconds. Multiple computer systems can perform audits in parallel such that the audit of the entire Optimal Chain can be quickly completed.

The audits can be performed automatically by computer systems at any time regularly or irregularly. If any problem is detected during any audit, the central computer system 1000 stops the module production and linking process such that Art Passion can investigate and fix the problem. The central computer system 1000 resumes its standard operational process after the problem has been fixed.

If there is a need to recover the data of a specific module in the database, the central computer system 1000 can copy the data of the same specific module stored inside the read-only memory and save the data into the database.

Because the only way to change the data stored inside the read-only memory is to physically replace the read-only memory with another piece of hardware containing a different set of data, Optimal Chain is immune to remote attacks by hackers because the remote hackers cannot appear in person to replace the read-only memory.

In one configuration of the present disclosure, Art Passion can use multiple computer systems, where each one is similar to the computer systems described above. These multiple computer systems may run in parallel to establish their respective Optimal Chains and constantly compare the module hash value of each module produced by the computer systems. If all computer systems have produced the same module hash value, they can continue to move forward because nothing is wrong. If the computer systems have different module hash values, they should stop, investigate, then fix the problem. This approach ensures error-free operations.

In one configuration of the present disclosure, Art Passion can allocate these multiple computers systems at multiple hidden locations which are far away from one another. This approach ensures that Optimal Chain cannot be modified.

Because a hacker does not physically show up at any location to replace the read-only memory, hackers cannot tamper with Optimal Chain. Therefore, Optimal Chain is a fraud-proof and hacking-proof electronic record system.

Although multiple computers are used to establish error-free, fraud-proof, and hacking-proof operations, the operational cost of Optimal Chain is much lower than the operational cost of Blockchain. It is more effective to use several computer systems to form Optimal Chain than to use thousands of computer systems to form Blockchain. As a result, Optimal Chain only consumes a very small amount of electrical power.

Although Art Passion is used as an example to explain how Optimal Chain works, in addition to artwork, Optimal Chain can be used for many different applications.

For example, if the artwork on Art Passion is replaced with the title document of a house, the Optimal Chain technology can record and track each historical purchase transaction of the house. As a result, people know exactly who owns the house at any particular time.

For example, if the artwork on Art Passion is replaced with the ownership of a car, the Optimal Chain technology can record and track each historical purchase transaction of the car. As a result, the Department of Motor Vehicle (DMV) knows exactly who owns the car at any particular time.

For example, if the artwork on Art Passion is replaced with the ownership of a virtual currency, the Optimal Chain technology can record and track each historical trading transaction of the virtual currency. As a result, people know exactly who owns the virtual currency at any particular time.

For example, if the transactional records of a bank are stored on Optimal Chain, the Optimal Chain technology can record and track each historical transactional record. As a result, people know exactly what transactions have happened in history.

For example, if the artwork on Art Passion is replaced with a stock certificate of a company, the Optimal Chain technology can record and track each historical trading transaction of the stock certificate. As a result, people know exactly who owns the stock certificate at any particular time.

For example, if the artwork on Art Passion is replaced with an electronic gift card, the Optimal Chain technology can record and track the ownership of the electronic gift card and each of the historical payment transactions of the electronic gift card. As a result, people know exactly who owns the electronic gift card and how much money is left on the electronic gift card at any particular time.

For example, if the artwork on Art Passion is replaced with a stored-value card (e.g., prepaid card), the Optimal Chain technology can record and track the ownership of the stored-value card (e.g., prepaid card) and each historical purchase transaction of the stored-value card (e.g., prepaid card). As a result, people know exactly who owns the stored-value card (e.g., prepaid card) and how much money is left at the stored-value card (e.g., prepaid card) at any particular time.

Therefore, Optimal Chain provides a reliable, effective, efficient, continuous, infinite, immutable, auditable, chronological electronic record system for all types of electronic files. Below is an example to illustrate how Optimal Chain handles an electronic file of any type.

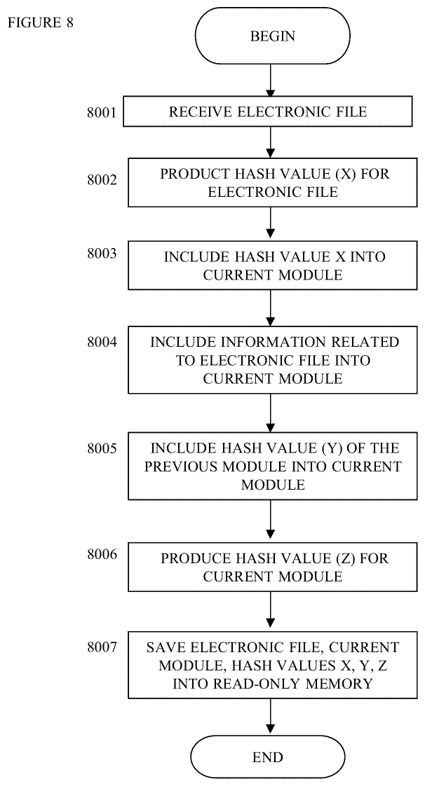

In one configuration, as shown in FIG. 8 together with FIG. 1C at block 8001, a central computer system (e.g., a device interface) receives an electronic file uploaded from a user of Optimal Chain.

At block 8002, the central computer system uses a hash function to produce a hash value for the uploaded electronic file. For ease of reference, we will refer to this hash value as hash value X.

At block 8003, the central computer system includes the hash value X into the contents of the current module (e.g., Nth module).

At block 8004, the central computer system also includes other information (e.g., time stamp, user identification information, user description, file identification information, file description, notes, comments, transactional details, etc.) related to the uploaded electronic file into the contents of the current module (e.g., Nth module).

At block 8005, the central computer system includes the hash value of the previous module (e.g., (N−1)th module) into the contents of the current module (e.g., Nth module). For ease of reference, we will refer to this hash value as hash value Y.

In addition to the specific electronic file mentioned above in this example, many other electronic files and information can also be included into the current module (e.g., Nth module).

Optimal Chain adds a new module to the chain periodically. The frequency of adding a module can be adjusted from time to time based on the need of the application. When it is time to add the current module (e.g., Nth module) to Optimal Chain, at block 8006, the central computer system uses the hash function to produce a hash value for the contents of the current module (e.g., Nth module). For ease of reference, we will refer to this hash value as hash value Z, which will be included as the contents of the next module (e.g., (N+1)th module).

At block 8007, the central computer system saves the electronic file, the current module (e.g., Nth module), hash values X, Y, and Z, and other related information into a read-only memory (e.g., backup computer system, Blu-ray disc, CD, DVD, or other similar read-only memory devices).

In the above example, Optimal Chain has created a reliable, effective, efficient, continuous, infinite, immutable, auditable, chronological electronic record for the electronic file. Hash values X, Y, and Z can be used for auditing by the computer automatically. If any of these hash values in the database are wrong during an audit, the central computer system can recover the original records from the read-only memory (e.g., backup computer system, Blu-ray disc, CD, DVD, or the other similar read-only memory devices).

Optimal Chain can be used to manage the issuance and maintenance of electronic financial instruments. FIG. 1D illustrates an example of Cheer Pay, which is an electronic gift card issuance and management system 1500 (e.g., a device interface) based on the Optimal Chain technology according to aspects of the present disclosure.

Cheer Pay is an organization that uses technologies to unite consumers and businesses in a local community so that they can jointly make the local community more prosperous.

Gift cards are often used for marketing promotions. However, gift cards have been targeted in various fraud schemes over the years. Here are some common types of fraud involving gift cards:

Impersonation Scams: Scammers often pose as government officials, tech support agents, or even relatives in distress. They claim the victim owes money or needs to pay for an urgent service and instruct the victim to purchase gift cards and share the card numbers and PINs. This is particularly common with scammers posing as IRS agents in the U.S., demanding back taxes be paid via gift cards-a request the real IRS would never make.

Resale Scams: After selling a legitimate gift card, the scammer, if they have retained the card number and PIN, might quickly spend the balance before the purchaser can use it.

Card Skimming: Criminals use devices or software to collect data from the magnetic stripe of a gift card from display racks in stores. They then monitor or check the card's balance online and, once loaded, quickly use or sell the card's value.

Return Fraud: Individuals purchase merchandise with stolen credit cards and then return the items to get the value on a gift card.

Employee Misconduct: Dishonest store employees might manipulate gift card activations, charging customers without properly loading the cards or using previously activated cards for new customer purchases and pocketing the difference.

Cheer Pay issues and manages electronic gift cards for local businesses and keeps all transactional records in Optimal Chain. As a result, common types of fraud cases involving gift cards are eliminated by Cheer Pay.

In one configuration, the computer network 1600 in FIG. 1D can be the Internet. A consumer uses his device interface 1700 (e.g., a mobile phone) to purchase and use the electronic gift card issued by the device interface 1800 (e.g., a mobile phone) of a business and managed by Cheer Pay 1500. As a result, three (3) computer systems in FIG. 1D have achieved the goal of issuing and managing electronic gift cards through the computer network 1600.

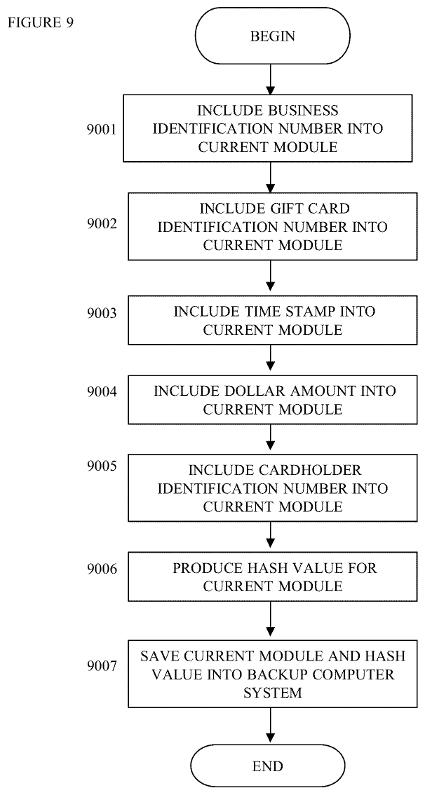

In one configuration, as shown in FIG. 9 together with FIG. 1D, the consumer's device interface 1700 (e.g., a mobile phone) sends through the computer network 1600 a request to the central computer system 1500 (e.g., a device interface) to issue an electronic gift card for the business which has a mobile phone 1800 (e.g., a device interface). The computer system 1900 is a backup computer system, which can be configured to become the read-only memory in the structure of Optimal chain.

First, the central computer system 1500 (e.g., a device interface) creates a unique gift card identification number to issue a new gift card for the business. The business is identified by a unique business identification number. The consumer is identified by a unique cardholder identification number. The issuance of a new gift card is a type of transaction. All data elements associated with the issuance transaction are linked with a unique transaction identification number in the database.

At block 9001, the central computer system 1500 includes the business identification number into the contents of the current module of Optimal Chain. There are many ways to implement this step. In this example, the central computer system 1500 has a database to store data. Therefore, the central computer system 1500 stores the module identification number of the current module “together with” the business identification number of the business into the database. For clarification purposes, when a first data element is stored together with a second data element, the first data element and the second data element are linked together in the database. This means that a database user can immediately find the second data element based on the first data element, and vice versa. This step enables the central computer system 1500 to find all modules containing the business identification number.

At block 9002, the central computer system 1500 includes the unique electronic gift card identification number into the contents of the current module of Optimal Chain by storing the module identification number of the current module together with the electronic gift card identification number into the database. This step enables the central computer system 1500 to find all modules containing the electronic gift card identification number.

At block 9003, the central computer system includes the time stamp (e.g., year, month, day, time, etc.) of the electronic gift card issuance into the contents of the current module of Optimal Chain by storing the module identification number of the current module together with the time stamp of the electronic gift card issuance into the database. This step enables the central computer system 1500 to apply a hash function later on the contents of the current module through a unique sequence based on time stamps to produce a unique hash value for the current module.

A rule can be set to resolve the situation when two elements of the contents of the module have the same time stamp. For example, the element with an electronic gift card identification number will be given a higher priority in the sequence. If both elements have electronic gift card identification numbers, the element with a larger number will be given a higher priority. If neither element has an electronic gift card identification number, the element with a cardholder identification number will be given a higher priority in the sequence. Similar rules can be set based on the above examples. The goal of these rules is to ensure that a set of data elements will always be arranged in the same sequence based on the rules so that a hash function will convert the set of data elements into the same hash value.

At block 9004, the central computer system 1500 includes the dollar amount of the electronic gift card into the contents of the current module of Optimal Chain by storing the module identification number of the current module together with the dollar amount of the electronic gift card into the database. This step enables the central computer system 1500 to find all modules containing the dollar amount of the electronic gift card.

At block 9005, the central computer system 1500 includes the unique cardholder identification number into the contents of the current module of Optimal Chain by storing the module identification number of the current module together with the cardholder identification number into the database. This step enables the central computer system 1500 to find all modules containing the cardholder identification number.

At block 9006, the central computer system 1500 uses the hash function to produce a hash value for the current module of Optimal Chain. A set of rules is used to list the data of the contents of the current module in a predefined sequence so that the central computer system 1500 will always produce the same hash value for the current module of Optimal Chain regardless of how many times the hash function is performed on the contents of the current module.

At block 9007, the central computer system 1500 saves the contents of the current module and the hash value for the current module of Optimal Chain into the backup computer system 1900, which operates as the read-only memory device in the structure of Optimal Chain. In practice, the central computer system 1500 can save the contents of the current module and the hash value for the current module into any backup device, which will be set up for read-only purposes. To increase the security protection, the backup computer system 1900 can further save the contents of the current module and the hash value for the current module of Optimal Chain into another read-only memory (e.g., Compact Disc Read-Only Memory (CD-ROM), Digital Versatile Disc Read-Only Memory (DVD-ROM), backup computer system, Blu-ray Disc Read-Only Memory (BD-ROM), Masked ROM (MROM), Programmable Read-Only Memory (PROM), Erasable Programmable Read-Only Memory (EPROM), Electrically Erasable Programmable Read-Only Memory (EEPROM), Flash Memory, or other read-only memory).

This hash value of the current module and the contents of the current module will be used to perform audits by computer systems later. In addition, the central computer system 1500 informs the business, which has a mobile phone 1800 (e.g., a device interface), of the issuance of the electronic gift card.

As a result of the above process as shown in FIG. 9, the issuance of the electronic gift card to the cardholder becomes permanently recorded into Optimal Chain. Nobody will be able to modify this record.

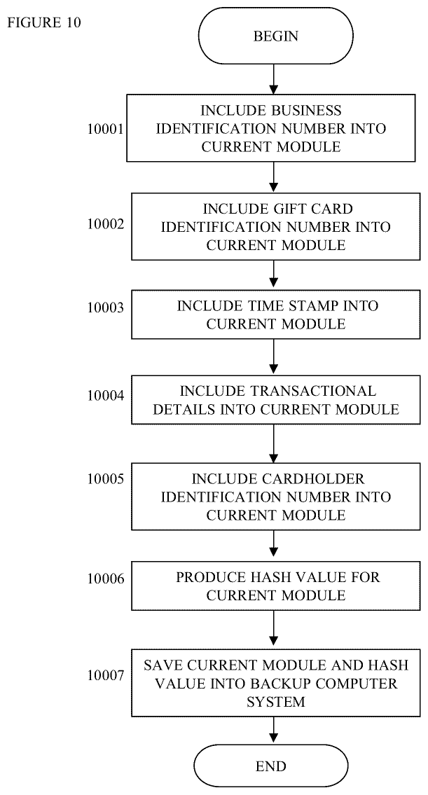

In one configuration, as shown in FIG. 10 together with FIG. 1D, the consumer's device interface 1700 (e.g., a mobile phone) sends through the computer network 1600 an instruction to the central computer system 1500 (e.g., a device interface) to conduct a payment transaction based on the gift card issued by the business, which has a mobile phone 1800 (e.g., a device interface). The computer system 1900 is a backup computer system that can be configured to become the read-only memory in the structure of Optimal chain. The payment is a type of transaction. All data elements associated with the payment transaction are linked with a unique transaction identification number in the database.

At block 10001, the central computer system 1500 includes the unique business identification number into the contents of the current module of Optimal Chain.

For clarification purposes, the inclusion of a data element into the contents of the current module of Optimal Chain is achieved by storing the module identification number of the current module together with the data element into the database such that the central computer system 1500 can find all modules containing the data element. This step also ensures that computer systems can conduct audits on the data integrity of Optimal Chain automatically.

At block 10002, the central computer system 1500 includes the unique electronic gift card identification number into the contents of the current module of Optimal Chain based on the approach explained above.

At block 10003, the central computer system 1500 includes the time stamp (e.g., year, month, day, time, etc.) of the electronic gift card transaction into the contents of the current module of Optimal Chain based on the approach explained above.

At block 10004, the central computer system 1500 includes the transactional details into the contents of the current module of Optimal Chain based on the approach explained above. The transactional details may include the balance of the electronic gift card before the transaction, the dollar amount of the transaction, the balance of the electronic gift card after the transaction, etc.

At block 10005, the central computer system 1500 includes the unique cardholder identification number into the contents of the current module of Optimal Chain based on the approach explained above.

At block 10006, the central computer system 1500 uses the hash function based on a set of rules to produce a hash value for the current module of Optimal Chain. The rules can be set by at least one of a person, a computer, or a combination thereof.

At block 10007, the central computer system 1500 saves the contents of the current module and the hash value for the current module of Optimal Chain into the backup computer system 1900, which operates as the read-only memory device in the structure of Optimal Chain. To increase the security protection, the backup computer system 1900 can further save the contents of the current module and the hash value for the current module of Optimal Chain into another read-only memory device (e.g., CD ROM, DVD ROM, PROM, backup computer system, Blu-ray Discs, etc.). In addition, the central computer system 1500 informs the business, which has a mobile phone 1800 (e.g., a device interface), of the transaction results of the electronic gift card.

As a result of the above process as shown in FIG. 10, the transactional record of the electronic gift card becomes permanently recorded into Optimal Chain. Nobody will be able to modify this record.

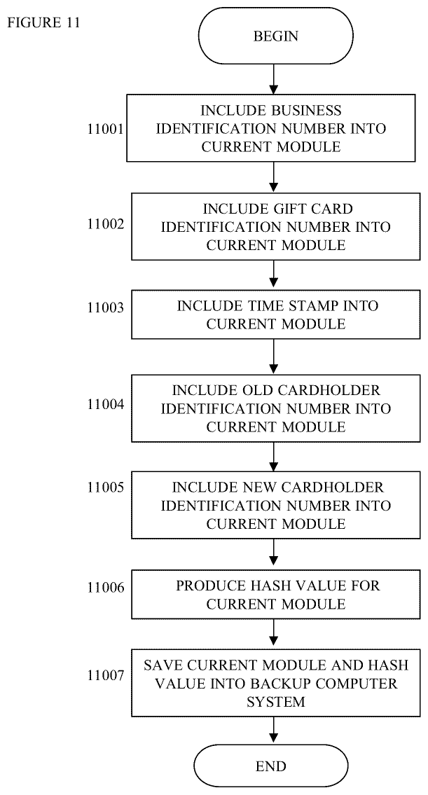

In one configuration, as shown in FIG. 11 together with FIG. 1D, the consumer's device interface 1700 (e.g., a mobile phone) sends through the computer network 1600 an instruction to the central computer system 1500 (e.g., a device interface) to change the ownership of the electronic gift card issued by the business, which has a mobile phone 1800 (e.g., a device interface). The computer system 1900 is a backup computer system that can be configured to become the read-only memory in the structure of Optimal chain. The ownership change is a type of transaction. All data elements associated with the ownership change transaction are linked with a unique transaction identification number in the database.

At block 11001, the central computer system 1500 includes the unique business identification number into the contents of the current module of Optimal Chain based on the approach explained above.

At block 11002, the central computer system 1500 includes the unique electronic gift card identification number into the contents of the current module of Optimal Chain based on the approach explained above.

At block 11003, the central computer system 1500 includes the time stamp

(e.g., year, month, day, time, etc.) of the electronic gift card transfer from an old cardholder to a new cardholder into the contents of the current module of Optimal Chain based on the approach explained above.

At block 11004, the central computer system 1500 includes the unique old cardholder identification number into the contents of the current module of Optimal

Chain based on the approach explained above.

At block 11005, the central computer system 1500 includes the unique new cardholder identification number into the contents of the current module of Optimal Chain based on the approach explained above.

At block 11006, the central computer system 1500 uses the hash function based on a set of rules to produce a unique hash value for the current module of Optimal Chain. The rules can be set by at least one of a person, a computer, or a combination thereof.

At block 11007, the central computer system 1500 saves the contents of the current module and the hash value for the current module of Optimal Chain into the backup computer system 1900, which operates as the read-only memory device in the structure of Optimal Chain. To increase the security protection, the backup computer system 1900 can further save the contents of the current module and the hash value for the current module of Optimal Chain into another read-only memory device (e.g., CD ROM, DVD ROM, PROM, backup computer system, Blu-ray Discs, etc.). In addition, the central computer system 1500 informs the business, which has a mobile phone 1800 (e.g., a device interface), of the transfer of the electronic gift card, the old cardholder of the electronic gift card, and the new cardholder of the electronic gift card.

As a result of the above process as shown in FIG. 11, both the old cardholder and the new cardholder of the electronic gift card become permanently recorded into Optimal Chain. Nobody will be able to modify this record.

For clarification purposes, the contents of a module are a set of data. The above examples illustrate how the central computer system 1500 handles a request for action from the computer system 1700; saves the action results into the backup computer system 1900, which operates as the read-only memory device of Optimal Chain; and informs the computer system 1800 of the result of the action requested by the computer system 1700. Additionally, a hash function is used by Optimal Chain to produce an immutable and auditable electronic record.

In one application of the present disclosure, a computerized method to produce an immutable and auditable electronic record comprises (1) receiving at a first computer system from a second computer system a request for an action on a data element; (2) conducting the requested action on the data element by the first computer system; (3) using a hash function to produce a hash value by the first computer system based on a set of data that contains a result of the requested action on the data element; (4) and sending from the first computer system to a third computer system the hash value and the set of data while the third computer system is used as a read-only memory device.

Adding a new module to Optimal Chain can be performed regularly or irregularly based on the needs of the application. For example, a new module can be added to Optimal Chain every second, or every 10 seconds, etc. Alternatively, for example, a new module can be added to Optimal Chain when a specific type of event has occurred while that type of event may occur irregularly.

When the central computer system 1500 conducts a simple audit on the data integrity of a specific module in the database, the computer system can compare the hash value for the specific module stored in the backup computer system 1900 with the hash value for the specific module stored in the database. If these two hash values are identical, the specific module has passed the simple audit.

When the central computer system 1500 conducts a comprehensive audit on the data integrity of a specific module in the database, the computer system can use the hash function to produce a “verification hash value” for the specific module in the database first, then compare the hash value for the specific module stored in the backup computer system 1900 with the verification hash value for the specific module in the database. If these two hash values are identical, the specific module has passed the comprehensive audit.

The above audits can be conducted automatically by the central computer system 1500 or another computer system. As a result, Optimal Chain ensures the data integrity of the data stored in the database.

The U.S. government requires financial institutions to conduct audits every year. It is extremely difficult for human beings to audit the integrity of electronic records in financial institutions because financial institutions may have millions of transactions per day. Most auditors only randomly select a specific period and a specific area to conduct audits because it is impossible to audit all the electronic records.

Most existing computer software systems used by financial institutions, only keep the most up-to-date data in the data fields and do not keep chronological records for the data fields. For example, the old balance of an account will be overwritten by the new balance of the account whenever there is a transaction occurring to the account. The old address of a customer will be overwritten by the new address of the customer whenever the customer moves to a new apartment. As a result, it is extremely difficult, if not impossible, to audit the integrity of electronic records.

Even for a limited audit scope, large financial institutions spend tens of millions of dollars to hundreds of millions of dollars every year on audits to comply with the regulatory requirements. Now, financial institutions can save a lot of money, resources, and time because financial institutions can use Optimal Chain to keep their electronic records so that their audits can be conducted by computers to cover all electronic records, instead of human beings who can only audit a small randomly selected scope of electronic records.

There are numerous existing computer software systems today. It is impractical to redevelop all these systems based on the Optimal Chain architecture. An alternative approach is to only move crucial electronic records to Optimal Chain so that the crucial electronic records can be automatically audited by Optimal Chain.

FIG. 1E illustrates an example of moving crucial electronic records, which will be produced by the existing software system in the future, to Optimal Chain so that Optimal Chain will produce immutable, auditable, chronological electronic records for the existing software system in the future. In this example, it is not possible to recover those old electronic records that were overwritten by the existing software system.

The first step is to identify a first database table (802, database table A in FIG. 1E), which contains the crucial electronic records produced by the original software program (801 in FIG. 1E) used by the existing software system.

The second step is to search the original software program (801) to find instructions that write data into database table A (802) and insert instructions in the original software program (801) to produce a revised software program (803 in FIG. 1E) so that whenever the original software program (801) writes a piece of data (X in FIG. 1E) into the first database table (802), the revised software program (803) writes the same piece of data (X) into both the first database table (802) and the new first database table (804 in FIG. 1E). To save the processing time of Optimal Chain, the time stamp of writing data into the new first database table (804) is also stored in the new first database table (804). The data in the new first database table (804) is used to form Optimal Chain (805 in FIG. 1E).

As a result, the revised software program (803) will maintain all the functions of the original software program (801). In addition, the revised software program (803) will duplicate the contents of the first database table (802) in the new first database table (804), which will keep the time stamped historical data in chronological order without overwriting.

The third step is to repeat the same process to modify the revised software program (803) for the second database table, the third database table, the fourth database table, etc., until the revised software program (803) has been modified to move all crucial electronic records to Optimal Chain. At the same time, the revised software program (803) has kept all the functions of the original software program (801).

Then, the original software program (801) is replaced by the revised software program (803) in the existing software system so that the existing software system will produce a new set of database tables that contain all the crucial electronic records to form Optimal Chain based on the process illustrated in FIG. 1C.

When it is time to form a current module for Optimal Chain, the computer system of Optimal Chain converts the crucial electronic records in the new set of database tables into the contents of the current module and produces a hash value for the contents of the current module based on the time stamps and table names stored in the new set of database tables. As a result, all crucial electronic records will be saved into Optimal Chain through the revised software program (803) running on the existing software system.

It is relatively easy to modify the original software program (801) into the revised software program (803). A simple conversion software program can be developed to perform such conversion so that the conversion may be completed by a computer accurately without human involvement.

The conversion software program may also include other techniques described in the present disclosure. For example, a set of instructions may be inserted into the revised software program (803) so that a hash value is produced for each electronic file and the hash value of each electronic file is stored into Optimal Chain. Consequently, nobody can modify any electronic file without being detected.

The above computerized method can quickly enhance existing computer software systems with Optimal Chain such that the existing computer software systems can also produce immutable and chronological electronic records that can be automatically audited by computer systems.

In one configuration of the present disclosure, Optimal Chain provides a reliable, effective, efficient, continuous, infinite, immutable, auditable, chronological electronic record system that can be used to record and track virtual currency trading, electronic art trading, or trading of any tangible or intangible subjects represented or managed by electronic records. For example, these electronic records may include ownership of at least one of a house, a building, a real estate a property, a land, a mine, a car, a boat, an airplane, a company, an organization, a club, a security, a loan, a right, an obligation, a physical property, an intellectual property, an electronic financial instrument, an electronic file, or a combination thereof.

Because Optimal Chain can keep electronic records immutable, it has broad applications across many industries. Described below are some of the possible applications:

Blockchain and Cryptocurrencies: Blockchain technology relies on maintaining an immutable ledger of transactions. Optimal Chain is an improved immutability technology and can enhance the security and reliability of blockchain systems and cryptocurrencies.

Financial Services: Banks and other financial institutions can use the Optimal Chain technology to keep secure, unalterable records of transactions, loans, and other financial activities. This can also help in auditing and regulatory compliance.

Healthcare: Because Optimal Chain keeps immutable records, it can enhance patient data management, ensuring the integrity of health records, test results, and treatment histories. This can be especially useful in research and in situations where multiple doctors need to access a patient's history.

Supply Chain Management: The Optimal Chain technology can maintain secure, unalterable records of product movement from manufacture to sale. This can help prevent fraud, theft, and counterfeit products.

Voting Systems: Because Optimal Chain is an immutable electronic record system, it can secure electronic voting results, ensuring that once a vote is recorded, it cannot be changed.

Education: Schools and universities can use the Optimal Chain technology to keep immutable records of student grades, degrees, and other achievements.

Real Estate: Because Optimal Chain keeps immutable records, Optimal Chain can enhance the security and reliability of property deeds, transaction histories, and other records related to real estate.

Legal Industry: Because Optimal Chain keeps immutable records, Optimal Chain can secure contracts, agreements, and other legal documents. Optimal Chain can also prove the authenticity of digital evidence.

Data Backup and Archiving: Because Optimal Chain keeps immutable records, Optimal Chain can improve the reliability of data backups and archives, ensuring that once data is stored, it cannot be changed or tampered with.

Internet of Things (IoT): In IoT, Because Optimal Chain keeps immutable records, Optimal Chain can provide secure and unchangeable logs of device status, activity, and transactions.

These are just some examples. The potential applications of Optimal Chain are vast, because many industries and sectors require secure, unalterable records

Some aspects of the present disclosure are directed to a regulatory compliance system, which empowers the operators of Optimal Chain to comply with laws and regulations.

For example, when the Optimal Chain technology is used for virtual currency trading, the operator of the specific Optimal Chain is classified as a money service business by the U.S. law. Therefore, the operator of Optimal Chain needs to comply with the Bank Secrecy Act (BSA) which was first established in 1970. Under the Bank Secrecy Act, money exchangers must report suspicious activities to the U.S. government. Suspicious activities cover a very broad scope. For example, money laundering, terrorist financing, fraud, embezzlement, identity theft, computer intrusion, self-dealing, bribery, false statement, counterfeit instruments, mysterious disappearance, etc., are all classified as suspicious activities.

After the 9/11 tragedy, U.S. lawmakers believed that effective compliance with the Bank Secrecy Act could have prevented the 9/11 tragedy. To further enforce the Bank Secrecy Act, the U.S. Congress passed the USA PATRIOT Act, which enacted severe civil and/or criminal penalties for violations of the Bank Secrecy Act.

Furthermore, the U.S. government agencies, such as Financial Crimes Enforcement Network (FinCEN), Office of Comptroller of Currency (OCC), Federal Reserve Bank (FRB), Federal Deposit Insurance Company (FDIC), National Credit Unions Administration (NCUA), State Banking Departments, Department of Financial Institutions, etc., strictly require financial institutions, including money services businesses, to comply with the Bank Secrecy Act, especially in their obligations to file Suspicious Activities Reports (SARs) to FinCEN.

In one configuration of the present disclosure, an intelligent alert system is used to monitor, detect, and report suspicious activities.

FIG. 1F illustrates an example of an intelligent alert system 500 (e.g., a device interface) and a computer network 600, such as a local area network, according to aspects of the present disclosure. In one configuration, the intelligent alert system 500 enables a BSA Officer 100, Compliance Officer 200, investigator 300 and Other Responsible Person 400 to comply with different types of laws and regulations and send SAR cases directly to another computer system 700 at FinCEN.

The Compliance Officer 200 configures and/or adjusts the parameters of the computer system 500 via the computer network 600. The computer system 500 uses an internal workflow function to send a potential case through the computer network 600 to the investigator 300. After the investigation, the investigator 300 sends the potential case and her investigation results through the computer network 600 to the computer system 500. The computer system 500 uses an internal workflow function to send the potential case and investigation results through the computer network 600 to the BSA Officer 100 for approval. After the BSA Officer 100 has approved the investigation results, if the potential case is a true positive, the computer system 500 receives the approval from the BSA Officer 100 through the computer network 600. Then, the computer system 500 sends the true positive to the computer system 700 at FinCEN.

In some financial institutions, the same person may have multiple job roles. For example, one person can be the BSA Officer, the Compliance Officer, and the investigator. Under such circumstances, the intelligent alert system uses its internal workflow function to assign different jobs to this person based on his different roles at different stages of the workflow.

After the computer system 500 has learned the experience of the investigator 300, the computer system 500 will become smarter and will automatically accept a potential case as a true positive if the conditional probability for the potential case to become a true positive is higher than a predefined value. Under such circumstances, the computer system 500 directly sends the true positive to the computer system 700 at FinCEN without any human involvement. The more the computer system 500 has been used by the investigator 300, the smarter the computer system 500 becomes. Over time, the computer system 500 will handle a majority, or all, of the potential cases by itself with little human involvement.

The intelligent alert systems can be installed at financial institutions, merchants, or any type of organizations which have the need to prevent financial crimes. More importantly, the intelligent alert system will learn from human beings and will become smarter to automatically accept potential cases as true positives and/or reject potential cases as false positives like human beings. As a result, the intelligent alert system can help a financial institution use minimum human resources to comply with different requirements, including laws, regulations, rules, etc.

In addition to the Bank Secrecy Act, the intelligent alert system can also help various organizations use minimum human resources to comply with many other laws and regulations through the monitoring of transactions and activities. Depending on the specific requirements of these laws and regulations, the intelligent alert system may monitor different types of activities by using different methods. The present disclosure provides various details of how to monitor transactions and activities and help various organizations use minimum human resources to comply with different types of requirements, laws, and regulations. Moreover, the intelligent alert system can also be used for other applications, or other organizations for other purposes. The intelligent alert system reduces or eliminates human efforts and mistakes, saves resources and money, and effectively achieves improved results.

Once the intelligent alert system has detected a potential financial crime against an individual (or an organization), the intelligent alert system sends the description of the financial crime and the contact information of the individual (or the organization) to a consumer protection system. A consumer protection system contacts the individual (or the organization) and requests the contacted party to verify whether it is truly a financial crime. The consumer protection system sends feedback from the individual (or the organization) to the intelligent alert system. The intelligent alert system uses the feedback to stop the financial crime if it is a true financial crime, or dismiss the potential case as a false positive if it is not a true financial crime.

Moreover, because the consumer protection system communicates with many intelligent alert systems residing at different financial institutions, the consumer protection system will receive feedbacks from many individuals and organizations which are customers of the financial institutions. This feedback information is also important to some third parties, such as merchants, who want to prevent future losses caused by similar financial crimes. These third parties have the desire to subscribe to the services provided by the consumer protection system which is capable of providing feedback information.

The consumer protection system has incorporated an alarm system and the subscribers are third parties, such as merchants. Based on the feedback from the individual (or the organization), the consumer protection system can send an alarm to the subscribers if there is a true crime. The third parties will use the information to stop future crimes so that the criminal or the fraudster cannot commit any similar crime against the individual or the organization anymore.

Many anti-money laundering professionals have come to realize that transactional monitoring cannot identify all money launderers. After obtaining the illicit proceeds, criminals often move to another financial institution to start over, carte blanche. These criminals pretend to be good citizens and financial institutions cannot detect any of their transactions as suspicious even though their illicit proceeds have been deposited into financial institutions. No transactional monitoring system can detect a money launderer who has no suspicious transactions.

For example, Financial Institution A's customer John Doe in Los Angeles disappears after he defaults on a $250,000 unsecured loan obtained through fraudulent misrepresentation. Then, $250,000 with no trace to Financial Institution A is deposited into an account at Financial Institution B in San Francisco that John Doe opened several years ago. If Financial Institution A knows that the illicit proceeds of John Doe are in Financial Institution B, it can seize the illicit proceeds in John Doe's account at Financial Institution B through a prejudgment writ of attachment.

However, the Gramm-Leach-Bliley Act in the U.S. and similar laws in other countries have prohibited financial institutions from disclosing non-public personal information of their customers or members. As a result, Financial Institution A cannot disclose John Doe's name in public and Financial Institution B cannot know that John Doe has stolen money from Financial Institution B.

The present application also discloses an illicit proceeds tracking system which tracks John Doe without disclosing any personal identification information of John Doe. In the above scenario, when Financial Institution A tracks the illicit proceeds of John Doe, Financial Institution B will receive an alert. Based on Section 314(b) of the USA PATRIOT Act, Financial Institution A and Financial Institution B are fully protected by the safe harbor when they discuss John Doe. Financial Institution A can obtain a prejudgment writ of attachment from the court to seize John Doe's funds in Financial Institution B.

Therefore, in addition to identifying the money launderers that are missed by the AML transactional monitoring system, Illicit Proceeds Tracking can substantially increase the overall profitability of a financial institution.

Moreover, if every financial institution tracks the illicit proceeds after a perpetrator has committed a financial crime, such as money laundering, terrorist financing, Ponzi scheme, human trafficking, embezzlement, bank fraud, security fraud, insurance fraud, tax fraud, etc., based on the Money Laundering Control Act which covers hundreds of Specified Unlawful Activities, the perpetrator cannot launder the illicit proceeds through any financial institution. This is the definitive goal of the anti-money laundering laws, regulations, and rules. The illicit proceeds tracking system will achieve this goal.

The U.S. government strictly enforces a business's compliance with the USA PATRIOT Act, the Bank Secrecy Act (BSA), the Fair and Accurate Credit Transactions Act (FACT Act), the Unlawful Internet Gambling Enforcement Act (UIGEA), the Elder Abuse Reporting Act, (EARA), the Sarbanes-Oxley Act (SOX), the regulations set by the Office of Foreign Assets Control (OFAC), and other related laws and regulations. The business may include, for example, financial institutions, such as banks, credit unions, mortgage companies, money services businesses, stockbrokers, and insurance companies. Billions of dollars in Civil Monetary Penalties (CMPs) have been levied by the U.S. government to financial institutions for violating these laws and regulations. Criminal penalties have also been issued to some individuals that work for the financial institutions.

A financial institution is just one type of business. Financial institutions are not the only organizations that need to comply with these laws and regulations. Many other types of businesses need to comply with these laws and regulations. The present disclosure applies to all businesses, such as businesses that are obligated to comply with laws and regulations.

The Financial Crimes Enforcement Network (FinCEN) and the Office of Foreign Assets Control (OFAC) are USA organizations. The laws and regulations in the USA are used as examples in the present disclosure. Many other countries have similar organizations that perform similar tasks. As such, similar laws and regulations exist in many other countries. The present disclosure is also applicable in those countries to help businesses comply with their respective laws and regulations. Aspects of the present disclosure may also be used by businesses, individuals, or organizations that do not need to comply with a law or regulation.

Often, it may be difficult to determine whether a person or a group of persons have conducted an illegal activity. According to the Bank Secrecy Act in the USA, when a business files a Suspicious Activity Report (SAR) with FinCEN, the business has no obligation to prove whether the reported case is an illegal activity. In fact, a “safe harbor” rule encourages businesses to report more suspicious activities without concern for the ramifications of being accused of reporting legitimate activities as wrongful by mistake. Under this “safe harbor” rule, no person (or organization) can file a lawsuit against an entity because the entity filed a Suspicious Activity Report (SAR) to FinCEN about this person (or organization). A SAR is used by the government to collect information, and a business is only expected to provide information and opinions in a SAR. Government agencies perform their own investigations to determine whether an activity reported in a SAR is truly illegal.

In general, the decision-making process regarding whether to report a suspicious activity, which is not fraud, is different from the decision-making process regarding whether to report a fraud case. For a fraud case, an entity, such as a business or a consumer, may lose money. Therefore, fraud is easier to identify in comparison to other crimes. As such, it is easier to decide whether to report a fraud case. Preventing fraud is also easier in comparison to preventing other crimes. As an example, if a computer system detects a high fraud risk associated with a transaction, the computer system can block the transaction and let an investigator investigate the transaction to determine whether it is truly a case of fraud.

In one aspect of the present disclosure, for fraud detection, a computer system calculates a risk score associated with a transaction based on different factors associated with the transaction. These factors may include historical activities of the account, deviations from the expected activities, location, time, amount, frequency and nature of the transaction, relationships between multiple accounts, type, nature, and structure of the account holder, etc.

In one aspect of the present disclosure, for fraud detection, a computer system blocks the transaction if the fraud risk score of the transaction is over a threshold. The threshold can be pre-determined based on the policy of the business.

In one aspect of the present disclosure, for fraud detection, a computer system creates a case based on the detected high fraud risk transaction. The case and related information are presented to an investigator for further investigation.

In comparison to fraud, there may be no clear evidence for suspicious activity. For example, a customer may frequently deposit a large amount of cash. It is possible that this customer is engaged in money laundering by selling illegal goods and receiving cash as payment. It is also possible that this customer sells homemade products in farmers' markets and only accepts cash as payments. Often, due diligence is needed to determine whether there is anything suspicious.

It is also possible that although a customer sells homemade products in farmers' markets, the customer also sells illegal goods at other locations. Unless the bank is informed that the customer sells illegal goods, there is no evidence for the bank to prove that the customer sells illegal goods. If the customer really sells illegal goods and the bank does not report such suspicious activity to FinCEN, the bank may receive a severe penalty later for failing to report the case to FinCEN once the customer is caught by the government for selling illegal goods.

On the other hand, if the bank reports every case that has a slight chance of being suspicious, the bank may invite unnecessary attention from government agencies. The government agencies may spend many months inside the bank to investigate the bank's operations and may severely affect the bank's operations.

The decision for reporting a case may be a judgment call by the person who reviews the case. In addition, the decision-making process can be quite subjective. Furthermore, a business cannot block a transaction simply because it appears to be a suspicious money laundering activity. A consumer may sue the business that blocks the consumer's transaction when the business cannot really prove that money laundering has occurred. In fact, many government agencies often advise businesses, which have reported suspicious activities such as money laundering or terrorist financing, to keep quiet and process the suspicious transactions as normal transactions so that the suspect will not be alerted and flee. This approach gives government agencies more time and opportunity to identify all related perpetrators.

According to the U.S. Bank Secrecy Act, a business that files a SAR has the obligation to keep the SAR confidential and cannot let the suspect (e.g., a person involved in the case) know anything about the SAR, including the existence of the SAR. SARs can only be reviewed by authorized government agencies.

Because handling a suspicious activity case is very different from handling a fraud case, as described above, many conventional approaches and concepts that are applicable for fraud detection and prevention are no longer useful for detecting and managing suspicious activity, such as money laundering, terrorist financing, elder abuse, online gambling, etc. In one aspect of the present disclosure, a computer system records the opinions of the person that decides not to report a detected suspicious activity case. Under such circumstances, the decision maker records a reason to justify their decision.

Unlike a fraud case, a suspicious activity case may not be clear to a person who reviews the case until additional evidence becomes available. Therefore, it is possible that a person may dismiss a detected suspicious activity case at the beginning, but change his mind later when additional evidence becomes available. In one aspect of the present disclosure, a person who reviews a detected suspicious activity case may also need to review all the historical detected cases regarding the same suspect to determine whether any new evidence, when combined with the old evidence, possibly from any dismissed cases, makes the newly detected case more suspicious. As a result, even if a case was previously dismissed as a false detection, such a dismissed case may be reviewed later.

This case review practice of suspicious activity may be different from the case review practice of fraud, as fraud cases typically have a clear conclusion. If a customer is a fraudster, the customer's account is closed and the customer will be prevented from conducting future transactions/activities. If a customer is a victim of fraud, the detected fraud case has nothing to do with the customer and the evidence is not used against the customer in the future. Therefore, a fraud investigator usually only focuses on newly detected cases. Conversely, a suspicious activity investigator may need to review a history of the detected cases and make a decision after intensive research and analyses. In one aspect of the present disclosure, the justification of the decision for not reporting a suspicious activity is stored in a database and is available for future reference.

In another aspect of the present disclosure, a computer system also records the identity of the person that decides not to report the detected case. The computer system may compare the decisions made by multiple persons for not reporting suspicious activities of the same suspect(s) to determine whether an investigator is attempting to hide a detected suspect or case.

For a large business, thousands of suspicious activities may be detected every month. A group of people may be tasked with reviewing the detected cases to determine whether the business needs to file SARs on these cases. In one aspect of the present disclosure, a computer system automatically allocates detected cases to different people based on the policies set by the business. The computer system may monitor and record the status of each detected case. If a case review is delayed by a particular person, the computer system will alert the business of such a delay.

In yet another aspect of the present disclosure, a computer system monitors the workload of each person who reviews detected cases. If a person has reviewed an unusually large number of cases in comparison to other persons who also reviewed detected cases during the same period of time, this person himself may become suspicious or questionable.

On the other hand, if a person has reviewed a small number of cases in comparison with other persons who also reviewed cases during the same period of time, this person may also become suspicious or questionable. In either of the above two situations, a manager of the business may want to investigate the situation and reach his own conclusion and resolution.

In general, different detection functions are used to detect suspicious activities because suspicious activities may arise in many different types of activities. Because the detection of suspicious activities is not clear, some detected cases may not be truly suspicious after investigation. Under such circumstances, such detected cases are dismissed as false detections or false positives. A false detection or false positive is generally referred to as the conclusion of an investigation of a case, but not the reason to justify why the case is dismissed.

For example, if a financial institution detects a case where several customers live at the same address and deposit a large amount of cash into the financial institution, this case may be about a possible drug dealer family, with many of the family members depositing their proceeds from selling drugs. After investigation, however, this case may actually be a group of students living together and depositing tips that they receive from working at a restaurant. The reason to justify the decision not to report this case should be “students living together are depositing tips they have received from part-time jobs.” As such, due to the given reason, the conclusion of the detected case becomes a false detection or false positive.

In general, after the review of a detected case, the case can be classified as a false detection (or false positive) by the person who reviewed this case. In one aspect of the present disclosure, a computer system provides information and/or statistics for a user to analyze all detected cases that have been classified as false detections. From these false detections, the user may identify detection functions that have generated a number of false detections that is greater than a threshold. The user can further improve the identified detection functions to improve the detection of future suspicious activities.

There are several methods to report SAR cases to FinCEN. One approach is to send the SAR report in electronic format directly to the server located at FinCEN. Under such circumstances, a BSA Officer can instruct the computer system that has detected suspicious activities to file a SAR report. The computer system will prepare the SAR report based on the suspect and the transactions identified by the BSA Officer, then transmit the SAR report to the computer system at FinCEN.

In one aspect of the present disclosure, a computer system compares a transactional pattern of a customer (or a group of customers) with known money laundering transactional patterns to detect suspicious money laundering activities. If there is a match, a possible money laundering activity may have been detected.

For example, many perpetrators know that a bank has to file a Currency Transaction Report (CTR) to the U.S. government if more than $10,000 in cash is deposited into a bank account on the same day. To avoid the filing of CTRs, perpetrators often split one large cash deposit into multiple smaller cash deposits, each cash deposit occurring on a different day and each cash deposit being less than $10,000. This transactional pattern is called “structuring,” a known money laundering transactional pattern, and a computer system can detect this type of transactional pattern. There are many other types of transactional patterns that are known as money laundering transactional patterns. A computer system can be designed to detect each of these known money laundering transactional patterns. As a result, even if there is no change of behavior, a money laundering activity can be detected based on the transactional pattern of the suspect or suspects.

In one aspect of the present disclosure, the BSA Officer (or responsible person) investigates the detected case to determine whether it is a true money laundering case. In one aspect of the present disclosure, the BSA Officer also reviews all historical cases associated with the suspect(s) of the currently detected case. In one aspect of the present disclosure, if the BSA Officer agrees that such transactions are suspicious activities, the computer system assists the BSA Officer to file a SAR to FinCEN. In another aspect of the present disclosure, if the BSA Officer decides not to file a SAR, the BSA Officer enters a reason into the computer system to justify his decision for not reporting such detected activities.

In another aspect of the present disclosure, a group of customers with one or more common risk factors (or characteristics), such as a type of business, business model, organization structure, size, location, product, service, career type, position, etc., are compared together to detect suspicious money laundering activities. If a transactional activity (e.g., transactional pattern, transactional volume, transaction frequency, transaction trend, number of transactions, amount of transactions, transaction derivative, etc.) of one customer is different from the transactional activities of other customers, this customer may have conducted a suspicious money laundering activity. In one aspect of the present disclosure, statistical figures such as mean, variance, standard deviation, percentile, etc., of the group of customers are used to facilitate such comparison. Similarly, if one customer behaves differently from other customers who have the same set of risk factors (or characteristics), this customer may have conducted a suspicious money laundering activity. As a result, even if there is no change in behavior in any account, a suspicious money laundering activity can be detected.

Sometimes, it is not easy to compare a group of customers together. For example, an MSB with 100 branches may have many more cash activities than another MSB with only two branches. In one aspect of the present disclosure, to achieve a more effective comparison, it is useful to compare some derivatives (e.g., ratio of several numbers) instead of the original raw data. For example, a ratio can be “total cash withdrawal from a bank divided by total number of checks deposited into a bank.” In this example, the number of checks deposited can be used to measure the size of the MSB's check-cashing operations. Therefore, the ratio “total cash withdrawal divided by total number of checks deposited” basically scales the check cashing operations of the 100-branch MSB and the check cashing operations of the two-branch MSB to about the same level, based on check-cashing activities so that they can be compared on a more level ground.

Many other derivatives can be used to achieve a better comparison. In general, a derivative for a more effective comparison may include “a first variable of interest divided by a second variable which measures the size of the business (or operations).” For example, “total ACH outgoing transactional amount divided by total number of checks deposited,” “total wire outgoing transactional amount divided by total number of checks deposited,” “total number of prepaid cards issued divided by total number of checks deposited,” “total ACH outgoing transactional amount divided by total number of branches,” “total wire outgoing transactional amount divided by total number of branches,” “total number of prepaid cards issued divided by total number of branches,” “total ACH outgoing transactional amount divided by total number of prepaid cards issued,” “total wire outgoing transactional amount divided by total number of prepaid cards issued,” etc., are just some examples of the possible derivatives that can be used. In one aspect of the present disclosure, in addition to the ratios above, other forms of mathematical transformation create a derivative.

In one aspect of the present disclosure, a computer system compares a derivative of a particular customer with the derivatives of a group of customers who have one or more common risk factors (or characteristics) with the particular customer (e.g., same type of business or profession). If the derivative of the particular customer noticeably deviates from the derivatives of the group of customers, the particular customer may have conducted a suspicious money laundering activity. In one aspect of the present disclosure, statistical analysis such as mean, variance, standard deviation, percentile, etc., of the group of customers facilitate such comparison.

In one aspect of the present disclosure, a computer system uses many different risk factors to determine the money laundering risk of each customer of a financial institution. For example, these risk factors may include an industry, category of the customer, business type of the customer, geographical area of the customer, country of the address of the customer, a nature of the customer's business product types of the business, services types of the business, structure of the business, profession of the customer, nationality, historical records (including the compliance records such as the number of Currency Transactional Reports, the number of Suspicious Activity Reports, the matches with the OFAC list, the matches with the 314(a) list, the matches with the Political Exposed Persons list, special designations by the compliance program, etc.), type of the transaction conducted, balance of the accounts, funds inflow, funds outflow, transactional pattern, number of transactions, amount of transactions, transactional volume, transactional frequency, transactional derivative, location of the transaction, time of the transaction, country of the transaction, sender of a money transfer transaction, location of the sender, country of the sender, nature of the sender, recipient of a money transfer transaction, location of the recipient, country of the recipient, nature of the recipient, relationships, social status, political exposure, historical transactions, etc. In fact, thousands of risk factors may be considered to determine the money laundering risk of a customer. For the purpose of this disclosure, a “risk factor” is also referred to as a “representative element of a risk dimension,” or simply a “risk dimension.”

According to aspects of the present disclosure, each attribute of a customer that can affect the risk of the customer is a risk factor. Additionally, each characteristic of a customer that can affect the risk of the customer may be a risk factor. Furthermore, each type of activity of a customer that can affect the risk of the customer is a risk factor. The risk factors may also be affected by other risks, such as a piece of information related to a customer, each type of transaction of a customer, and/or each transactional pattern of a customer. Each risk factor is assigned a risk value.

In one configuration, each degree of the same type of risk is a risk factor and is given a risk score. For example, a total cash transactional amount in a period of 30 days can be used to measure the degree of the risk associated with money laundering. For example, we can define a total cash transactional amount level (or total cash transactional amount degree) from $0 to $5,000 during a 30-day period to have a risk score of 10; from $5,001 to $50,000, a risk score of 50; from $50,001 to $250,000, a risk score of 100; from $250,001 to $1,000,000, a risk score of 200; from $1,000,001 to $10,000,000, a risk score of 500; and for $10,000,000 and above, a risk score of 1,000. In this example, a person with a total cash transactional amount of $60,000 during a period of 30 days is classified into the amount level of “between $50,001 and $250, 000,” and has a risk score of 100.

“Cash transaction amount” is just used as an example. Other considerations, such as number of cash transactions, acceleration of cash transaction, etc., can also be used to measure the degrees of risks associated with money laundering. In addition to cash, other financial transactions such as checks, wires, ATM, ACH, virtual currency, virtual securities, virtual instruments, credit card, debit card, prepaid card, monetary instruments, transfer, etc., can also be used to measure the degrees of risks associated with money laundering. Those skilled in the art can easily figure out numerous risk factors based on the above example.

In one aspect of the present disclosure, a risk score-based scenario is based on customer data. Each piece of information about the customer is a risk factor and is assigned a risk score. Additionally, or alternatively, a risk score-based scenario is based on transactional data. Each amount level (or amount degree) of a type of transaction is a risk factor and is assigned a risk score.

In one aspect of the present disclosure, customer data is associated with one or more of an industry category of the customer, a business type of the customer, a geographical area of the customer, a country of an address of the customer, a nature of a business of the customer, a product type of the business, a services type of the business, a structure of the business, a profession of the customer, a nationality of the customer, a historical record, a type of the transaction conducted, a balance of an account, funds inflow, funds outflow, a transactional pattern, a number of transactions, an amount of transactions, a transactional volume, a transactional frequency, a transactional derivative, a location of the transaction, a time of the transaction, a country of the transaction, a sender of a money transfer transaction, a location of the sender, a country of the sender, a nature of the sender, a recipient of a money transfer transaction, a location of the recipient, a country of the recipient, a nature of the recipient, a relationship, social status, political exposure, a historical transaction, a number of suspicious activity reports (SARs) filed for money laundering and terrorist financing cases, a category of a first financial institution, a business type of the first financial institution, geographical area of the first financial institution, country of a head office of the first financial institution, nature of the business of the first financial institution, age of a person, sex of the person, income level of the person, appearance of the person, judgment about the person, a personal condition of the person, a family condition of the person, a family member of the person, a family member's condition of the person, a friend of the person, a friend's condition of the person, a historical record of the person, an industry category of the person, a geographical area of the person, a country of an address of the person, a profession of the person, a job type of an employee, an education level of an employee, an income level of an employee, a length of employment at a current job, a performance review record, employment history, a duration of each employment in the employment history, a reason for termination of each employment in the employment history, an age of the employee, a sex of the employee, a personal condition of the employee, a family condition of the employee, a family member of the employee, a family member's condition of the employee, a friend's condition of the employee, a historical record of the employee, a type of work performed, a number of transactions performed, an amount of transactions performed, a largest amount of transaction, a number of transactions with a particular counter party, an amount of transactions with a particular counter party, a number of changes of a crucial record, a number of changes of a crucial record associated with a particular counter party, a geographical area of an employee's home, a geographical area of an employee's office, a country of the address of the employee, a due diligence result of the customer, a length of an account history, a number of name matches with gambling organizations in transactions, or a combination thereof.

In one aspect of the present disclosure, transactional data is associated with one or more of cash, check, wire transfer, ATM (Automated Teller Machine), ACH (Automated Clearing House), virtual currency, virtual securities, virtual instruments, credit card, debit card, prepaid card, electronic fund transfer, wires, monetary instruments, letters of credit, notes, securities, commercial papers, commodities, precious metal, account opening, account closure, an account application, deposit, withdrawal, cancellation, balance check, inquiry, credit, debit, or a combination thereof. The transactional data can be from real transactions of the customer or anticipated transactions provided by the customer in a customer due diligence process during account opening.

In one aspect of the present disclosure, each risk factor is given a risk score and a customer is given a total risk score, which is a summation of all risk scores of the risk factors associated with the customer. This process of producing a total risk score for each customer may be referred to as risk scoring. This total risk score is used to determine the level of risk associated with the customer. A summation is used as one example in this disclosure. In fact, many different types of mathematical transformations can also be used to achieve a similar effect.

In one aspect of the present disclosure, each risk factor is given a risk score and a customer is given a total risk score, which is a value derived from a mathematical transformation of all risk scores of the risk factors associated with the customer.

As explained earlier, unlike a fraud situation, a higher-risk client may not be a suspect for money laundering or terrorist financing. High risk may just be the nature of the client. For example, MSBs, pawnshops, car dealers, pilots, flight attendants, etc., are often classified as higher-risk customers for anti-money laundering and anti-terrorist financing purposes, however, this does not mean that these customers are conducting money laundering activities or terrorist financing.

Nevertheless, because a customer has a high-risk score, the customer may be closely monitored and a different monitoring method may be applied. The different methods may include using more due diligence effort, time, data, information sources, questions, etc., during the investigation process. Therefore, in one aspect of the present disclosure, the total risk score of a customer is used to determine the monitoring method applied to monitor the customer. If the total risk score of a customer is higher, a closer monitoring method is applied to monitor the customer. More due diligence will be conducted on higher-risk customers whose total risk scores are higher than the value determined based on group statistics of a group of customers. If the total risk score of a customer is lower, a more relaxed monitoring method is applied to monitor the customer. During continuous monitoring of a customer, if the total risk score of the customer has increased more than a threshold, the customer may have conducted suspicious activity. In some aspects of the present disclosure, for higher-risk customers, the threshold should be smaller so that the higher-risk customers are monitored more closely than lower-risk customers.

In other words, in some aspects of the present disclosure, a total risk score of a customer is not used to determine whether the customer is suspicious. Instead, a total risk score of a customer is used to select an algorithm or a set of algorithms for monitoring the customer or a method for conducting more due diligence during the investigation of detected suspicious activities. If the total risk score of a customer is higher than a threshold during the customer due diligence process at account opening, enhanced due diligence should be performed. An enhanced due diligence process means that more questions, data, information resources, research, analyses, investigations, site visits, etc., will be used than with the standard due diligence process.

Sometimes, a customer with a very high risk score may be suspicious. Therefore, in one aspect of the present disclosure, if the total risk score of a customer is higher than a predefined value, an alert about the customer will be triggered so that the investigator can investigate the potential case. The predefined value can be set by a software module, a person who designs the system, a person who tunes the system, a person who uses the system, or a combination thereof.

In one aspect of the present disclosure, a group of customers having the same risk factor are compared together. For example, we can compare all the customers who are flight attendants together. In one aspect of the present disclosure, if the total risk score of a particular flight attendant is much higher than a reference value derived from the total risk scores of all flight attendants, this particular flight attendant may have conducted some suspicious money laundering activities. The reference value comprises an average, a median, a mean, a mode, a weighted average, and/or other statistical values.

Statistical approaches can also be applied to facilitate the detection of suspicious activities. For example, mean, variance and standard deviation can be derived from the total risk scores of all the customers who are flight attendants. In one aspect of the present disclosure, if the total risk score of a particular flight attendant is higher than the mean of total risk scores of all flight attendants by over 4 times the standard deviation, this particular flight attendant may have conducted suspicious activities.

The above reference “4 times” is just one example. The number “4” can be any number, such as 3.75, 4.21, 10, etc. In one aspect of the present disclosure, if the total risk score of a particular flight attendant is higher than the mean of total risk scores of all flight attendants by over x times of the standard deviation, this particular flight attendant may have conducted suspicious money laundering activities, where x is a number assigned by the BSA Officer (or a responsible person). This statistical approach can be applied whenever a group comparison is used.

A flight attendant is merely one example to illustrate this method to detect suspicious money laundering activities among a group of entities. In practice, many other risk factors can be used for similar purposes. Because there are tens of thousands of risk factors, in one aspect of the present disclosure, a computer system permits a user to select any risk factor to identify all customers who have the same risk factor. In one aspect of the present disclosure, if a particular customer has a total risk score which is much higher than a reference value derived from the total risk scores of other customers who have the same risk factor, the particular customer may have conducted suspicious money laundering activities. The reference value comprises an average, a median, a mean, a mode, a weighted average, a percentile, and/or other statistical values. The 50th percentile is in the middle of a distribution. When a total risk score of an entity is in higher percentiles, the total risk score of the entity is higher than 50% of the total risk scores of the group of entities.

Instead of one risk factor, a group of risk factors can also be used. In fact, a group of risk factors may improve the accuracy of the detection results. For example, in addition to the risk factor of profession (e.g., flight attendant), the destination country of the flight on which the flight attendant works can be another useful risk factor to detect money laundering risk. For example, a flight attendant who works on a flight between New York and Chicago may have activities, which are different from the activities of another flight attendant who works on a flight between Miami and Mexico City. It may be more accurate to compare a sub-group of flight attendants who work on flights between Miami and Mexico City. In this example, two risk factors, profession and destination city of flight, are considered to improve the accuracy of detection.

In one aspect of the present disclosure, a set of risk factors is used to identify a group of entities. If a particular entity has a total risk score that is much higher than a reference value derived from the total risk scores of all entities having the same set of risk factors, the particular entity may have conducted suspicious money laundering activities. The reference value comprises an average, a median, a mean, a mode, a weighted average, and/or other statistical values. To simplify the calculations, the standard group statistics such as mean, variance, standard deviation, etc., which can be easily calculated based on existing software developing tools, can be derived to facilitate such comparison among a group of entities. As a result, even if there is no behavior change in any account, a computer system can still detect suspicious money laundering activities based on the above approach.

Sometimes, it may be helpful to eliminate some entities from the group comparison process because such entities are very different from others. In one aspect of the present disclosure, a computer system permits users to select some entities that will not be included in a group comparison process.

Detecting a flight attendant as having a suspicious money laundering activity is just one example. A similar method can be applied to many other different situations. For example, it is usually very difficult for banks or credit unions to detect a Money Services Business (MSB) customer as having suspicious money laundering or terrorist financing activity because an MSB has many transactions every day and one money laundering transaction may be hidden among many other normal transactions.

In one aspect of the present disclosure, an additional risk factor (e.g., near Mexico border) is used to identify a group of MSBs (e.g., in addition to the first risk factor-type of business) which have this same set of risk factors. If a particular MSB has a total risk score higher than a reference value derived from the total risk scores of all MSBs having the same set of risk factors, the particular MSB may possibly have conducted suspicious money laundering activities. The reference value comprises an average, a median, a mean, a mode, a weighted average, and/or other statistical values. Similarly, standard group statistics such as mean, variance, standard deviation, etc., can be derived to facilitate such comparison among a group of MSBs.

Sometimes, it is not easy to compare a group of MSBs because they may have different types of operations and different sizes. In one aspect of the present disclosure, a part-time MSB and a full-time MSB are given two different risk factors because they may have different natures of business. In another aspect of the present disclosure, each of the different types of MSB products and/or services is given a risk factor. For example, each money transfer, check cashing, currency exchange, prepaid card management, etc., is given a risk factor, although all of them can be offered by the same MSB. In one aspect of the present disclosure, a set of risk factors that precisely define the types of products and/or services is used to identify the risks.

In one aspect of the present disclosure, some risk factors are adjusted based on the size of the operations so that a group comparison becomes more effective. For example, an MSB with 50 branches may naturally have 5 times the total cash transactional amount of another MSB with 10 branches. Sometimes, to conduct a group comparison, risk factors affected by the size of the operations may be adjusted to account for the size of the operations. For example, for an MSB with 50 branches, its total cash transactional amount in 30 days can be divided by 50 to establish an adjusted risk factor and a risk score for a group comparison. Branches are used here as an example to measure the size of the operations. Other information, such as number of customers, number of transactions, number of employees, size of assets, etc., can also be used to measure the size of the operations.

In one aspect of the present disclosure, a set of risk factors, adjusted based on the size of operations (e.g., adjusted risk factors), are used to identify a group of entities which have this set of adjusted risk factors. The risk score of an adjusted risk factor is referred to as the adjusted risk score. If a particular entity has a total adjusted risk score that is much higher than a reference value derived from the total adjusted risk scores of all entities having the same set of adjusted risk factors, the particular entity may have conducted suspicious money laundering activities. The reference value comprises an average, a median, a mean, a mode, a weighted average, and/or other statistical values. In general, in one aspect of the present disclosure, a detection algorithm that incorporates a risk factor in the detection algorithm can also be modified to incorporate an adjusted risk factor in the detection algorithm. A detection algorithm that incorporates a risk score in the detection algorithm can also be modified to incorporate an adjusted risk score in the detection algorithm.

To simplify the calculations, standard group statistics such as mean, variance, standard deviation, etc., based on adjusted risk factors and adjusted risk scores can be derived to facilitate such comparison among a group of entities. As a result, even if there is no behavior change in any account, a computer system can still detect suspicious money laundering activities based on the above approach.

Because MSBs may have different transactional activities from other types of businesses, it is more effective to monitor MSBs based on their unique transactional activities. Therefore, in one aspect of the present disclosure, a different set of detection algorithms may be used to monitor entities with a different set of risk factors. In one aspect of the present disclosure, a set of risk factors is used to identify a group of entities that have this set of risk factors, and a particular set of detection algorithms are used to detect suspicious money laundering activities in this group of entities. In other words, a set of detection algorithms is selected based on a set of risk factors associated with a group of entities to monitor the group of entities.

In another aspect of the present disclosure, a set of risk factors is adjusted based on the size of operations and used to identify a group of entities that have this set of adjusted risk factors, and a particular set of detection algorithms are used to detect suspicious money laundering activities in this group of entities. In other words, a set of detection algorithms is selected based on a set of adjusted risk factors associated with a group of entities to monitor the group of entities.

Sometimes, it is meaningful to monitor entities with higher risks more closely than entities with lower risks. Therefore, different sets of detection algorithms are used to monitor different entities with different levels of risk. In one aspect of the present disclosure, a set of detection algorithms is selected based on a total risk score of an entity to monitor the entity. In another aspect of the present disclosure, a set of detection algorithms is selected based on a total adjusted risk score of an entity to monitor the entity where the total adjusted risk score is obtained from the risk scores of adjusted risk factors.

In one aspect of the present disclosure, once an MSB is detected as having possible money laundering activities, a computer system can identify the transaction (or a group of transactions) that caused the detected MSB to have a higher total risk score than a reference value derived from the total risk scores of all MSBs. The reference value comprises an average, a median, a mean, a mode, a weighted average, and/or other statistical values.

Similarly, once an MSB is detected as having possible money laundering activities, a computer system identifies the transaction (or a group of transactions) that caused the detected MSB to have a higher total adjusted risk score than a reference value derived from the total adjusted risk scores of all MSBs. The reference value comprises an average, a median, a mean, a mode, a weighted average, and/or other statistical values. As a result, a money laundering transaction (or a group of money laundering transactions) can be identified through this approach. This approach of identifying a particular transaction (or a group of transactions) with a higher risk score (or higher adjusted risk score) can be used for other types of customers, not just for MSBs.

Conventionally, a higher risk score implies a higher risk. There is no rule, however, that prohibits a person or business from defining a lower risk score for a higher risk. To avoid confusion, the description in this disclosure is based on the convention that a higher risk score means a higher risk. Furthermore, a risk score can be a negative value. A negative risk score implies a reduced risk based on this convention.

As described above, an MSB is just one example. Other types of businesses, e.g., pawn shops, car dealers, etc., can be monitored in a similar way. As a result, risk factor, risk score, adjusted risk factor, adjusted risk score, total risk score, and total adjusted risk score can be used in a variety of methods to detect suspicious money laundering activities even if there is no behavior change in any account.

Sometimes, a bank receives a wire transfer from a client of corresponding bank A and resends the wire transfer to another client of corresponding bank B because corresponding bank A and corresponding bank B do not have a direct banking relationship. This situation often arises during international wire transfers because banks in two different countries may not have a direct banking relationship. This type of wire transfer is often referred to as intermediary wire transfer.

A bank that provides intermediary wire transfer services is exposed to a very high money laundering risk because senders and recipients of intermediary wire transfers are not customers of the bank. In addition, the bank may not know the real background of the senders and the recipients of the wire transfers. It is possible that a sender is a terrorist financier and a recipient is the terrorist. A bank that handles intermediary wire services may unknowingly become a channel for money laundering and terrorist financing.

In one configuration of the present disclosure, a computer system compares names, addresses, countries, phone numbers, email addresses, etc., of all senders and recipients of intermediary wire transfers and identifies the transactions associated with each sender and each recipient. In one aspect of the present disclosure, if the computer system detects an unusually large number of wire transfers from the same sender, the sender and the recipients may be involved in money laundering or terrorist financing activities. If the computer system detects an unusually large aggregate amount of wire transfers from the same sender, the sender and the recipients may be involved in money laundering activities.

Similarly, if the computer system detects an unusually large number of wire transfers to the same recipient, the senders and the recipient may be involved in money laundering or terrorist financing activities. If the computer system detects an unusually large aggregate amount of wire transfers to the same recipient, the senders and the recipient may be involved in money laundering activities.

If the computer system detects that an unusual number of wire transfers are sent from the same sender to the same recipient, the sender and the recipient may be involved in money laundering or terrorist financing activities. If the computer system detects that an unusual aggregate amount of wire transfers is sent from the same sender to the same recipient, the sender and the recipient may be involved in money laundering or terrorist financing activities.

In one aspect of the present disclosure, a BSA Officer investigates such a detected case to determine whether it is a true money laundering case. The BSA Officer also reviews all historical cases associated with the suspects of the newly detected case. If the BSA Officer agrees that there is suspicious money laundering activity, the computer system assists the BSA Officer to file a SAR to the FinCEN. If the BSA Officer decides not to file an SAR, the BSA Officer enters a reason into the computer system to justify his decision for not reporting such detected activities.

The Office of Foreign Assets Control (OFAC) has a very simple rule stating that it is illegal to have any business transaction with any entity on the list published by the Office of Foreign Assets Control. This list is commonly referred to as the “OFAC List.” This rule applies to all U.S. persons and entities, including financial institutions. For example, Wal-Mart was fined by the OFAC for a violation of this rule. U.S. financial institutions that are under the tightest regulatory monitoring naturally must strictly comply with this rule.

In the beginning, it was a very simple rule. The implications of this rule, however, have become much more complicated over the past 20 years. A common issue occurs when people misspell (including mistype, mispronounce, etc.) their names. Even if an entity's name was misspelled but is on the OFAC List, a financial institution still has the obligation of identifying this entity as an entity on the OFAC List (generally referred to as an OFAC match).

The natural question is how much deviation from the original names on the OFAC List is classified as “misspelling.” The OFAC and government regulators have never given any precise guidance for answering this question. A very common exercise an examiner or auditor may perform is to use a notorious name like “Osama bin Laden” as a sample to test a business. In general, a business is supposed to identify all business transaction associated with “Osama bin Laden,” “Osama Laden,” “Osama Laten,” “Laten Osama,” “Latin Obama,” etc., as possible OFAC matches. Now, if the scope of deviation from the OFAC names is further broadened, whether a financial institution is supposed to identify a single word “Obama” which is a former U.S. president's name as a possible OFAC match is questionable. One can easily tell that such a simple OFAC rule has created a great deal of confusion in recent years.

In one aspect of the present disclosure, an “OFAC Match Scale” is used to measure the degree of deviation. A value referred to as “Relative Correlation” (“RC value”) may be generated by the OFAC Match Scale to measure the similarity between two names. For example, if a name has an RC value of 100%, it exactly matches an OFAC name on the OFAC list. If a name has an RC value of 97%, it may be different from an OFAC name on the OFAC list by one letter or two letters. If a name has an RC value of 0%, it is totally different from all OFAC names on the OFAC list.

In one aspect of the present disclosure, the length of the name also affects the RC value. For example, if a name is different by one letter from an OFAC name, which has 25 letters, the RC value may be 96%, while another name may have an RC value of 90% although it is also different by just one letter from another OFAC name, which has 10 letters.

Some long words, such as international, incorporation, limited, company, organization, etc., are commonly used for business names, and such words also exist in the OFAC names list. As a result, these long words generate higher RC values for those businesses that use these long words in their names. To avoid unnecessary false positives, in one aspect of the present disclosure, a commonly used long word may be replaced with a short word to reduce its impact on the RC value. For example, the word “international” can be replaced with “intl.”

In addition, some countries do not use the descriptions “first name” and “last name.” As a result, a person may use a different sequence of names when this person is asked to provide a first name and a last name. “Osama Laden” may become “Laden Osama.” In one aspect of the present disclosure, an OFAC match scale identifies a possible “off-sequence” OFAC match.

Moreover, some words are commonly used in certain cultures without contributing distinct differentiations. For example, “bin” means “son of” and “binti” means “daughter of” in the Muslim culture. Official names in the Muslim culture have either “bin” or “binti” in the name. For example, if a Muslim father has the name “John,” his daughter “Mary” will have an official name of “Mary binti John” and his son “David” will have an official name “David bin John.” Under such circumstances, the words “bin” and “binti,” which are commonly used in Muslin names, will create “false similarity” between two Muslim names. To provide a more scientifically correct result, in one aspect of the present disclosure, the OFAC match scale may exclude these kinds of “trivial words” before the calculation of the RC value. Sometimes, a name may be translated into English based on sound. Therefore, in one aspect of the present disclosure, the OFAC match scale should measure the match of sound to determine the RC value.

In one aspect of the present disclosure, a financial institution decides what threshold to use when it conducts an OFAC check. For example, if a financial institution uses a threshold of 75%, a possible OFAC match is detected when a name has an RC value of 75% or higher. Because each financial institution may have a different risk exposure from others, it is very likely that X is the best threshold for financial institution A while Y is the best threshold for financial institution B. As a general guideline, the X or Y value is selected in accordance with a risk-based principle.

In general, the higher threshold a financial institution uses, the fewer possible OFAC matches the financial institution detects. This saves time during the review process because more false positives are avoided. If the threshold is too high, however, it is possible that the financial institution may miss a reasonable deviation from an OFAC name, such as “Osama bin Laden.” If the threshold is too low, it is possible that the financial institution may falsely detect many of its clients as possible OFAC matches. The best practice is to find a tradeoff between “too many possible OFAC matches to review” and “missing real OFAC name deviations caused by misspellings.”

In one aspect of the present disclosure, a user can randomly select a number of OFAC names from the OFAC List and find out how the OFAC match scale responds to deviations from these selected OFAC names. A user can then determine when he calls a “possible OFAC match” based on this test. It is advisable to keep this test result for future auditors and examiners to review.

There is a possibility that a particular name is very close to an OFAC name. For example, American Express, a very reputable credit card company, is often falsely detected as an OFAC match because of the word “express.” Therefore, to avoid this type of frequent false positive, in one aspect of the present disclosure, an exempt list is generated by users to include those well-known reputable businesses on the exempt list. The businesses on the exempt list are classified as false positives, either automatically by the computer or manually by users, when they are detected as possible OFAC matches.

Very often, a business may have an OFAC officer that handles all OFAC related matters. In one aspect of the present disclosure, if an OFAC Officer (e.g., a responsible person) of a financial institution detects a possible OFAC match with an RC value over a pre-defined threshold, the OFAC Officer investigates whether this is a true OFAC match. If the OFAC Officer believes that it is a true match, the OFAC Officer should handle the case according to the guidelines issued by the Office of Foreign Assets Control. According to the OFAC regulations, in some cases, the OFAC Officer may need to block the transaction so that the person on the OFAC list does not benefit from the transaction. If the OFAC Officer decides that the OFAC match is a false positive after his investigation, the OFAC Officer should enter a reason into the computer system to justify his reason for not reporting such OFAC match case to the Office of Foreign Assets Control and/or not block the transaction.

Section 314(a) of the USA PATRIOT Act requires financial institutions to detect matches of names on a 314(a) list, which is periodically published by the FinCEN. The computer system can handle the 314(a) compliance matters using a similar approach to the handling of the OFAC compliance matters, as described above.

Sometimes, a 314(a) list also includes additional personal identification information, such as identification document number, date of birth, address, etc., In one aspect of the present disclosure, in addition to the methods described above for detecting possible OFAC matches, personal identification information, such as identification document number, address, and/or date of birth, etc., is used by the computer system to determine whether a detected 314(a) match is a true match. This approach can reduce false positives in the 314(a) matching process.

In one aspect of the present disclosure, if a Compliance Officer (e.g., a responsible person) of a financial institution detects a possible 314(a) match with an RC value over a pre-defined threshold, the Compliance Officer investigates whether this is a true 314(a) match. In one aspect of the present disclosure, if the Compliance Officer believes it is a true match, the Compliance Officer reports the 314(a) match case to the FinCEN. If the Compliance Officer decides that the 314(a) match is a false positive after his investigation, the Compliance Officer enters a reason into the computer system to justify his reason for not reporting the 314(a) match to the FinCEN.

In one aspect of the present disclosure, the computer system receives customer information and transactional data from the core data processing system of the financial institution or other data processing systems that may be internal to or external from the financial institution. The customer information may include background information.

In one aspect of the present disclosure, the computer system receives information regarding suspicious activities observed by frontline personnel. For example, the computer system may receive information input from the frontline personnel. The computer system may also receive information provided by other internal or external sources.

Although “financial institutions” are used as examples for easy explanation, the present disclosure also applies to other types of businesses. In general, any business that needs to comply with laws and regulations may employ an intelligent alert system as described in the present disclosure.

In one aspect of the present disclosure, the risk score of a risk factor or a degree of a risk factor can be assigned by a computer software module, a person who designs or tunes the system, or a user who uses the system. In most cases, the absolute value of the risk score is not crucial and the relative relationships among all risk scores may be more important.

Moreover, a subject's total risk score should only fluctuate within a reasonable range. In one aspect of the present disclosure, if a subject's total risk score has suddenly increased and is beyond a threshold, the subject may have conducted a suspicious or unusual activity. That is, if a difference between a subject's first total risk score and second total risk score is greater than an increase threshold, where the first total risk score is less than the second total risk score, the subject may have conducted a suspicious or unusual activity. In another aspect of the present disclosure, if a subject's total risk score has suddenly decreased tremendously, the subject may also have conducted a suspicious or unusual activity. That is, if a difference between a subject's second total risk score and first total risk score is greater than a decrease threshold, where the first total risk score is greater than the second total risk score, the subject may have conducted a suspicious or unusual activity. Therefore, an alert will be sent to an investigator, a BSA Officer, a Compliance Officer, or another type of responsible person who will investigate the subject when its total risk score has suddenly increased or decreased tremendously.

The observed data of a subject may fluctuate from time to time. Thus, the intelligent alert system may permit a certain range of fluctuation of the subject's total risk score to avoid false alerts. In one aspect of the present disclosure, the intelligent alert system increases a subject's permissible total risk score fluctuation range when the subject's total risk score is lower than a threshold. In another aspect of the present disclosure, the intelligent alert system decreases a subject's permissible total risk score fluctuation range when the subject's total risk score is higher than a threshold. The permitted fluctuation range may be determined (e.g., set) by a software module, a person who designs the system, a person who tunes the system, or a person who uses the system.

For example, if a subject's total risk score is higher than the mean of the total risk scores of all subjects plus a certain number of standard deviations of all risk scores, such as four standard deviations, the intelligent alert system may modify the subject's permissible total risk score fluctuate range to be within half a standard deviation without triggering an alert. In another example, if a subject's total risk score is within the mean of the total risk scores of all subjects plus a certain number of standard deviations, such as three standard deviations, the intelligent alert system may permit the subject's total risk score to fluctuate within a range of one standard deviation without triggering an alert.

In yet another example, if a subject's total risk score is within the mean of the total risk scores of all subjects plus a certain number of standard deviations, such as two standard deviations, the intelligent alert system may permit the subject's total risk score to fluctuate within a range of one and a half standard deviations without triggering an alert. In still yet another example, if a subject's total risk score is within the mean of the total risk scores of all subjects plus a certain number of standard deviations, such as one standard deviation, the intelligent alert system may permit the subject's total risk score to fluctuate within a range of two standard deviations without triggering an alert.

In the field of machine learning, a negative is a set of data that has not triggered an alert. A true negative is a set of data that has not triggered the alert and does not comprise a true case for triggering the alert. A false negative is a set of data that has not triggered the alert but comprises a true case for triggering the alert that the system has missed. As an example, a false negative money laundering case may cause a financial institution to be penalized by the U.S. government if the false negative case is discovered by the U.S. government. Therefore, it is desirable to prevent false negatives in an alert system specified to prevent money laundering (e.g., anti-money laundering alert system).

For an anti-money laundering alert system in a U.S. financial institution, a true money laundering case is reported to FinCEN, a U.S. government organization. FinCEN has a set of communication protocols. Financial institutions in the U.S. can report cases to FinCEN by sending a file from the anti-money laundering alert system to the computer system at FinCEN based on FinCEN's communication protocols.

Conventionally, rule-based systems are used to detect suspicious activities and each rule may trigger an alert. Many financial institutions have used the rule-based approach, which can trigger numerous alerts. For example, there are over two hundred countries in the world. If a financial institution uses a rule-based approach to monitor the wire transfers to, or from, each country, the financial institution may have over two hundred branches at the country decision node of the decision tree. As another example, there are thousands of different industries. If a financial institution uses the rule-based approach to monitor the wire transfers to, or from, each industry, the financial institution may have thousands of branches at the industry decision node of the decision tree. Country and industry are two of many risk categories that have money laundering risks. Similarly, a wire transfer is one of many types of transactions that have money laundering risk. For example, cash, check, ACH, ATM, credit card, debit card, letter of credit, etc., are other possible types of transactions.

There are many money laundering risk factors. There are numerous (e.g., millions) of possible combinations of branches to form a path from the root of a decision tree to the leaf nodes of the decision tree. In other words, a rule-based system may use millions of rules to cover the entire scope of money laundering risk to detect suspicious money laundering activities. A rule-based system with a limited number of rules may have an increased number of false negatives (e.g., the system has missed true money laundering cases) and many false positives (e.g., the leaf nodes of the decision tree have an increased number of impurities and cannot achieve the goal of classification). Due to the number of false negatives and false positives when a rule-based approach is used, financial institutions hire investigators to review a large number of alerts. It is difficult for financial institutions to mitigate all false negatives with a rule-based system.

In the field of machine learning, conventional systems consider an accuracy of 70% as satisfactory. It is difficult, if not impossible, to train a machine learning model to have high accuracy, such as 100% accuracy. Unfortunately, although the 70% accuracy may be good for some purposes, this 70% target cannot satisfy the regulatory standards, such as those set by the U.S. government. As discussed, a financial institution may receive a severe regulatory penalty if it does not detect certain activity, such as money laundering. As such, a financial institution will not use an alert system with 70% accuracy. Therefore, conventional machine learning models are not satisfactory for an intelligent anti-money laundering alert system.

According to aspects of the present disclosure, the intelligent anti-money laundering alert system uses a risk-scoring approach. Each risk factor or a degree of a risk factor may be similar to a branch in a rule-based system. As such, the risk scoring process for producing a total risk score from many risk factors, as described in the present disclosure, may consolidate the information from many rules into the total risk score. For example, if a total risk score is generated from 10,000 risk factors, a user only needs to pay attention to those alerts that have the total risk score over a threshold without the need to evaluate each of the 10,000 risk factors. If a rule-based approach is used, each risk factor may have two possible outcomes, matched or not-matched. The total number of possible combinations of outcomes for 10,000 risk factors is two (2) to the power 10,000 (e.g., 210,000). Therefore, an evaluation based on the total risk score has effectively replaced the need to evaluate each of the two (2) to the power 10,000 (e.g., 210,000) possible outcomes. Because these 210,000 outcomes could potentially generate 210,000 different types of alerts, the intelligent anti-money laundering alert system can avoid at least 210,000 alerts. Therefore, the intelligent anti-money laundering alert system is an improvement in view of the conventional rule-based system.

Although one total risk score can replace many rules, it may not replace all rules. For example, if a person frequently deposits a certain amount of cash (e.g., $9,900), which is slightly below the CTR reporting threshold of $10,000, the financial institution is expected to report this person to Financial Crimes Enforcement Network (FinCEN) as a structuring case. It is difficult to detect a structuring case accurately based on the total risk scores. Therefore, an alert system based on the risk score based technology may include some rules in addition to the criteria based on risk scores.

In one aspect of the present disclosure, the intelligent anti-money laundering alert system uses risk score-based scenarios to replace rules. In one example, the intelligent anti-money laundering alert system may use approximately twenty to thirty scenarios. The scenarios may include both risk score-based scenarios and non-risk score based scenarios.

In addition to, or alternate from, the scenarios, other conditions may be used to generate an alert. For example, a computer system, such as a machine learning network, may be trained to generate a model. After training, the discriminant used by the model may be converted into an if-then conditional format to trigger alerts.

For the purposes of this disclosure, a scenario may be defined as a condition or a set of conditions that can trigger an alert or can be used to classify a subject into a category for a particular purpose. For example, a customer with a total risk score within a certain range may not trigger an alert. Still, in this example, the total risk score can classify the customer into a specific risk category, such as high risk, medium risk, or low risk. As another example, a customer that was previously a suspect in a suspicious activity report (SAR) may not trigger an alert. In this example, the customer may be classified into a specific category, such as prior SAR suspect or another similar category. As another example, a customer matched with the OFAC list, the 314(a) list, the Political Exposed Person list, and/or other lists may be classified in one or more categories.

A scenario may consist of a rule, a set of rules, a criterion, or a set of criteria, based on rules, facts, behavior patterns, risk scores, risk dimensions, total risk scores, special categories, mathematic models, and/or machine learning models. The scenario may trigger an alert by using a rule-based method, a behavior-based method, a risk-based method, a model-based method, and/or a machine learning-based method (e.g., an artificial intelligence-based method). An intelligent alert system may include one or more scenarios.

As discussed, an alert may be triggered by a scenario. The scenario may be flagged when one or more conditions are satisfied. A potential case that has triggered an alert may be referred to as a positive. The potential case may include one or more alerts. Therefore, the cause for the potential case may be one or more scenarios. The potential case, or the positive, may be investigated. A true positive may refer to a potential case (e.g., positive) that is a true case. If the investigation indicates that the potential case is not a true case, the potential case may be referred to as a false positive. Consequently, the false positive may be dismissed and the associated alerts may be dismissed as false alerts. The true positive may be reported to an authority, such as FinCEN or law enforcement.

In one configuration, a posterior probability may be estimated via a Bayesian principle. The product of the posterior probability and evidence is a prior probability multiplied by class likelihood. Using the application of reporting suspicious money laundering activities to FinCEN as an example, the Bayesian equation is p(S/c)p(c)=p(c/S)p(S). The evidence p(c) is the probability of potential cases triggered by the cause c out of all potential cases. The class likelihood p(S) is the probability of the true positives S (e.g., true SAR cases) out of all potential cases. The prior probability p(c/S) is the probability of true positives triggered by the cause c out of all true positives. As a result, the posterior probability p(S/c) may be determined as follows: p(S/c)=p(c/S)p(S)/p(c). The posterior probability P(S/c) is also the conditional probability that a potential case triggered by the cause c is a true positive. That is, although the conditional probability P(S/c) is derived from the historical data, it is the best estimate for the future probability that a potential case triggered by the cause c will become a true positive. As such, the posterior probability may also be referred to as the conditional probability for the future, or future conditional probability.

In this disclosure, the terminology “encoding” generally refers to any type of data manipulation so that an alphanumeric code is generated from a set of data through this data manipulation. The terminology “decoding” generally refers to the reverse data manipulation to transform the “encoded” data back to its original format. Not all encoded information can be decoded. Some coding schemes compress the information contained in the data, such that some information is lost during the encoding process. Sometimes, encoding involves encryption which hides the information such that it cannot be decoded or otherwise made visible without access to a description key.

In this disclosure, the terminology “network” generally refers to a communication network or networks, which can be wireless or wired, private or public, real time or non-real time, or a combination of them, and includes the well-known Internet.

In this disclosure, the terminology “computer” or “computer system” generally refers to either one computer or a group of computers, which may work alone or work together to accomplish the purposes of the system.

In this disclosure, the terminology “processor” generally refers to either one processor or a group of processors, which may work alone or work together to accomplish the purposes of the processor.

In this disclosure, the term “module” refers to a single component or multiple components which can be hardware, software, firmware, or a combination thereof, and may work alone or work together to accomplish the purposes of the module.

In this disclosure, a “bank” or “financial institution” generally refers to a financial service provider, either a bank or a non-bank, where financial services and money services are provided. Some examples of financial institutions are banks, credit unions, insurance companies, insurance agencies, stockbrokers, stock agencies, bond brokers, bond agencies, commodity brokers, commodity agencies, securities companies, mortgage companies, mortgage agencies, securities companies, money services businesses, agencies for money services businesses, agencies for organizations that provide financial services or money services, financial holding companies, trading companies, trading agencies, other financial service providers, other financial agencies, stock exchanges, commodity exchanges, securities exchanges, currency exchanges, virtual currency companies, virtual currency issuers, virtual currency service providers, virtual currency network providers, virtual currency computer providers, virtual currency dealers, virtual currency exchanges, virtual securities exchanges, bond exchanges, other exchanges, funds managers, investment companies, private equity firms, venture capital firms, merchant acquirers, payment processors, payment card issuers, payment card program managers, internet merchants, transactions processors, securities processors, other organizations related to financial services, etc.

In this disclosure, a “bank account” or “financial account” generally refers to an account associated with a financial institution, either a bank or a non-bank, where financial transactions can be conducted through financial instruments such as cash, virtual currency, virtual instruments, virtual securities, checks, credit cards, debit cards, ATM cards, stored value cards, gift cards, prepaid cards, wires, monetary instruments, letters of credit, notes, securities, commercial papers, commodities, securities, precious metal, electronic fund transfers, automatic clearing house, etc.

In this disclosure, “financial transactions” generally refer to transactions related to financial activities, including but not limited to payment, fund transfer, money services, securities issuance, securities exchange, currency exchange, commodity exchange, payroll, invoicing, trading, escrow, insurance, underwriting, merger, acquisition, account opening, account closing, account status check, etc.

In this disclosure, “trading” generally refers to trading activities, both private and public, including but not limited to trading of stock, currency, virtual currency, virtual instruments, virtual securities, commodities, rights, values, securities, derivatives, goods, services, merchandise, etc.

In this disclosure, “securities” are generally referred to according to the definition in the Securities Act of 1933 and other laws and regulations related to the Securities Act of 1933. For example, securities may generally include note, stock certificate, bond, debenture, check, draft, warrant, traveler's check, letter of credit, warehouse receipt, negotiable bill of lading, evidence of indebtedness, certificate of interest or participation in any profit-sharing agreement, collateral-trust certificate, preorganization certificate or subscription, transferable share, investment contract, voting-trust certificate; valid or blank motor vehicle title; certificate of interest in property, tangible or intangible; instrument or document or writing evidencing ownership of goods, wares, and merchandise, or transferring or assigning any right, title, or interest in or to goods, wares, and merchandise; or, in general, any instrument commonly known as a “security”, or any certificate of interest or participation in, temporary or interim certificate for, receipt for, warrant, or right to subscribe to or purchase any of the foregoing.

In this disclosure, a “consumer” generally refers to a customer, person, subject, payer, payee, beneficiary, user, or client, etc., seeking to perform a transaction with an individual, an organization, a merchant, and/or a financial institution.

In this disclosure, the terminology “identification document” generally refers to a passport, driver's license, voter card, benefits card, student identification card, social security card, national identification card, identity card, certificate of legal status, and other official documents and information bearing instruments that identify a designated individual by certain verifiable characteristics, that are issued or certified by a consulate, embassy, government agency, public or private organizations or other governmental authorities, and that are protected against unauthorized copying or alteration by the responsible party or parties. In particular, such “identification documents” can be formed from various materials, including paper, plastic, polycarbonate, PVC, ABS, PET, Teslin, composites, etc., and can embed the identification information in various formats, including printed or embossed on the document (or card), written on a magnetic medium, programmed into an electronic device, stored in a memory, and combinations thereof. The “identification information” may include, but is not necessarily limited to, names, identification numbers, date of birth, signatures, addresses, passwords, phone numbers, email addresses, personal identification numbers, tax identification numbers, national identification numbers, countries that issue the IDs, states that issue the IDs, ID expiration date, photographs, fingerprints, iris scans, physical descriptions, and other biometric information. The embedded information can be read through optical, acoustic, electronic, magnetic, electromagnetic, and other media.

In this disclosure, “personal identification information” generally refers to name, address, date of birth, personal identification number, user ID, password, tax identification number, type of the identification document used, identity number associated with the identification document, country, state, government organization and/or a private organization issuing the identification document, expiration date of the identification document, phone number, screen name, e-mail address, photographs, fingerprints, iris scans, physical descriptions, biometrical information, and other information that can be used to identify a person.

In this disclosure, “personal information” includes personal identification information, personal relationships, personal status, personal background, personal interests, and personal financial information including information related to financial instruments, financial accounts and financial activities, and other information that is related to a person.

In this disclosure, “financial instruments” generally refer to instruments that are used to conduct financial transactions. Examples of financial instruments include cash, virtual currency, virtual securities, virtual instruments, credit cards, debit cards, ATM cards, prepaid cards, stored value cards, gift cards, checks, monetary instruments, wire transfers, ACH transfers, letters of credit, notes, securities, commercial papers, commodities, precious metal, gold, silver, etc.

In this disclosure, a “personal communication device” generally refers to a device interface used for personal communication purposes.

In this disclosure, a “device interface” generally refers to a keyboard, a keypad, a monitor, a display, a terminal, a computer, a control panel, a vehicle dash board, a network interface, a machinery interface, a video interface, an audio interface, an electrical interface, an electronic interface, a magnetic interface, an electromagnetic interface including an electromagnetic wave interface, an optical interface, a light interface, an acoustic interface, a video interface, an audio interface, a contactless interface, a mobile phone interface, a smartphone interface, a smartbook interface, a tablet interface, other communication device interface, a Personal Digital Assistant (PDA) interface, a handheld device interface, a portable device interface, a wireless interface, a wired interface, and other interfaces.

In this disclosure, the terminology “terminal” or “kiosk” generally refers to equipment, including a computer and/or its peripherals, microprocessor and/or its peripherals, ATM terminal, check cashing kiosk, money services kiosk, merchant checkout stand, cash register, coin exchange machine, parking lot payment kiosk, other payment kiosks, contactless device, wire line phone, mobile phone, smartphone, smartbook, tablet, personal communication device, tablet device, digital assistant, entertainment device, network interface device, router, and/or Personal Digital Assistant (PDA), etc., which interfaces a user with a computer network, so that the user may interact with computer systems and other equipment connected to the computer network.

In this disclosure, the terminology “computer application module” generally refers to a computer-based application, including a hardware application, a software application, or an application based on both hardware and software. A computer application module comprises Outlook, Gmail, Hotmail, Facebook, LinkedIn, or any application that is running on a mobile phone, a computer system, a personal computer, a smartbook, a personal communication device, a device interface, or a combination thereof.

In this disclosure, the terminology “communication channel” generally refers to a message communication channel, an email communication channel, a webmail communication channel, a secure email communication channel, a social network communication channel, a mobile app communication channel, a password-controlled communication channel, a virtual communication channel, a physical communication channel, an embedded communication channel, an electronic communication channel, an electromagnetic communication channel, a mechanic communication channel, a laser communication channel, a light communication, a video communication channel, an audio communication channel, or a combination thereof.

The methodologies described may be implemented by various means depending upon the application. For example, these methodologies may be implemented in hardware, firmware, software, or any combination thereof. For a hardware implementation, the processing may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described, or a combination thereof.

For a firmware and/or software implementation, the methodologies may be implemented with modules (e.g., procedures, functions, and so on) that perform the functions described. Any machine-readable medium tangibly embodying instructions may be used in implementing the methodologies described. For example, software codes may be stored in a memory and executed by a processor. Memory may be implemented within the processor or external to the processor. As used the term memory” refers to any type of long term, short term, volatile, nonvolatile, or other memory and is not to be limited to any particular type of memory or number of memories, or type of media upon which memory is stored.

If implemented in firmware and/or software, the functions may be stored as one or more instructions or code on a computer-readable medium. Examples include computer-readable media encoded with a data structure and computer-readable media encoded with a computer program. Computer-readable media includes physical computer storage media. A storage medium may be any available medium that can be accessed by a computer. By way of example, and not limitation, such computer-readable media can comprise RAM, ROM, EEPROM, CD-ROM, DVD, Blu-ray Discs, or other optical disk storage, magnetic disk storage or other magnetic storage devices, or any other medium that can be used to store desired program code in the form of instructions or data structures and that can be accessed by a computer; disk and disc, as used, includes compact disc (CD), laser disc, optical disc, digital versatile disc (DVD), floppy disk and Blu-ray disc where disks usually reproduce data magnetically, while discs reproduce data optically with lasers. Combinations of the above should also be included within the scope of computer-readable media.

In addition to storage on computer readable medium, instructions and/or data may be provided as signals on transmission media included in a communication apparatus. For example, a communication apparatus may include a transceiver having signals indicative of instructions and data. The instructions and data are configured to cause one or more processors to implement the functions outlined in the claims. The communication apparatus may not store all of the instructions and/or data on a computer readable medium.

The aspects described in this disclosure can be assembled to form a variety of applications based on the need. Those skilled in the art and technology to which this disclosure pertains can appreciate that alterations and changes in the described structure may be practiced without meaningfully departing from the principal, spirit, and scope of this disclosure. Such alterations and changes should not be construed as deviations from the present disclosure.

What is claimed is:

1. A computerized method to present a multimedia artwork, comprising:
receiving, at a first computer system, a video file from a second computer system;
receiving, at the first computer system, a scent control file from the second computer system;
sending, from the first computer system to a third computer system, the video file;
sending, from the first computer system to the third computer system, the scent control file;
instructing, by the first computer system, the third computer system to send the scent control file to a fourth computer system;
instructing, by the first computer system, the third computer system to run the video file; and
instructing, by the first computer system, the third computer system to synchronize the fourth computer to run the scent control file when the third computer system is running the video file.

2. The computerized method of claim 1, in which the first computer system comprises a device interface.

3. The computerized method of claim 2, in which the device interface comprises at least one of a keyboard, a keypad, a monitor, a display, a terminal, a computer, a control panel, a vehicle dashboard, a network interface, a machine interface, an electrical interface, an electronic interface, a magnetic interface, an electromagnetic interface including an electromagnetic wave interface, an optical interface, a light interface, an acoustic interface, a video interface, an audio interface, a contactless interface, a mobile phone interface, a smartphone interface, a smartbook interface, a tablet interface, another communication device interface, a Personal Digital Assistant (PDA) interface, a handheld device interface, a portable device interface, a wireless interface, a wired interface, or a combination thereof.

4. The computerized method of claim 1, in which the second computer system comprises a device interface.

5. The computerized method of claim 4, in which the device interface comprises at least one of a keyboard, a keypad, a monitor, a display, a terminal, a computer, a control panel, a vehicle dashboard, a network interface, a machine interface, an electrical interface, an electronic interface, a magnetic interface, an electromagnetic interface including an electromagnetic wave interface, an optical interface, a light interface, an acoustic interface, a video interface, an audio interface, a contactless interface, a mobile phone interface, a smartphone interface, a smartbook interface, a tablet interface, another communication device interface, a Personal Digital Assistant (PDA) interface, a handheld device interface, a portable device interface, a wireless interface, a wired interface, or a combination thereof.

6. The computerized method of claim 1, in which the third computer system comprises a device interface.

7. The computerized method of claim 6, in which the device interface comprises at least one of a keyboard, a keypad, a monitor, a display, a terminal, a computer, a control panel, a vehicle dashboard, a network interface, a machine interface, an electrical interface, an electronic interface, a magnetic interface, an electromagnetic interface including an electromagnetic wave interface, an optical interface, a light interface, an acoustic interface, a video interface, an audio interface, a contactless interface, a mobile phone interface, a smartphone interface, a smartbook interface, a tablet interface, another communication device interface, a Personal Digital Assistant (PDA) interface, a handheld device interface, a portable device interface, a wireless interface, a wired interface, or a combination thereof.

8. The computerized method of claim 1, in which the fourth computer system comprises a device interface.

9. The computerized method of claim 8, in which the device interface comprises at least one of a keyboard, a keypad, a monitor, a display, a terminal, a computer, a control panel, a vehicle dashboard, a network interface, a machine interface, an electrical interface, an electronic interface, a magnetic interface, an electromagnetic interface including an electromagnetic wave interface, an optical interface, a light interface, an acoustic interface, a video interface, an audio interface, a contactless interface, a mobile phone interface, a smartphone interface, a smartbook interface, a tablet interface, another communication device interface, a Personal Digital Assistant (PDA) interface, a handheld device interface, a portable device interface, a wireless interface, a wired interface, or a combination thereof.

10. A computerized method to maintain integrity of an electronic file, comprising:
receiving, at a first computer system from a second computer system, the electronic file;
creating, by the first computer system, a unique identification number for the electronic file;
using, by the first computer system, a hash function to generate a hash value for the electronic file;
saving, by the first computer system, the electronic file, the unique identification number, and the generated hash value together into a database; and
saving, by the first computer system, at least the unique identification number and the generated hash value into a read-only memory.

11. The computerized method of claim 10, in which the read-only memory comprises at least one of Compact Disc Read-Only Memory (CD-ROM), Digital Versatile Disc Read-Only Memory (DVD-ROM), backup computer system, Blu-ray Disc Read-Only Memory (BD-ROM), Masked ROM (MROM), Programmable Read-Only Memory (PROM), Erasable Programmable Read-Only Memory (EPROM), Electrically Erasable Programmable Read-Only Memory (EEPROM), Flash Memory, other read-only memory, or a combination thereof.

12. The computerized method of claim 10, in which the first computer system comprises a device interface.

13. The computerized method of claim 12, in which the device interface comprises at least one of a keyboard, a keypad, a monitor, a display, a terminal, a computer, a control panel, a vehicle dashboard, a network interface, a machine interface, an electrical interface, an electronic interface, a magnetic interface, an electromagnetic interface including an electromagnetic wave interface, an optical interface, a light interface, an acoustic interface, a video interface, an audio interface, a contactless interface, a mobile phone interface, a smartphone interface, a smartbook interface, a tablet interface, another communication device interface, a Personal Digital Assistant (PDA) interface, a handheld device interface, a portable device interface, a wireless interface, a wired interface, or a combination thereof.

14. The computerized method of claim 10, in which the second computer system comprises a device interface.

15. The computerized method of claim 14, in which the device interface comprises at least one of a keyboard, a keypad, a monitor, a display, a terminal, a computer, a control panel, a vehicle dashboard, a network interface, a machine interface, an electrical interface, an electronic interface, a magnetic interface, an electromagnetic interface including an electromagnetic wave interface, an optical interface, a light interface, an acoustic interface, a video interface, an audio interface, a contactless interface, a mobile phone interface, a smartphone interface, a smartbook interface, a tablet interface, another communication device interface, a Personal Digital Assistant (PDA) interface, a handheld device interface, a portable device interface, a wireless interface, a wired interface, or a combination thereof.

16. A computerized method to issue an electronic financial instrument, comprising:
receiving, at a first computer system from a second computer system, a request to issue the electronic financial instrument;
creating, by the first computer system, a unique identification number for the electronic financial instrument;
retrieving, by the first computer system, an identification number associated with the second computer system;
saving, by the first computer system, the unique identification number for the electronic financial instrument and the retrieved identification number associated with the second computer system into a database;
using, by the first computer system, a hash function to generate a hash value from a set of data, which includes at least the unique identification number for the electronic financial instrument and the retrieved identification number associated with the second computer system; and
saving, by the first computer system, the generated hash value and the set of data into a read-only memory.

17. The computerized method of claim 16, in which the read-only memory comprises at least one of Compact Disc Read-Only Memory (CD-ROM), Digital Versatile Disc Read-Only Memory (DVD-ROM), backup computer system, Blu-ray Disc Read-Only Memory (BD-ROM), Masked ROM (MROM), Programmable Read-Only Memory (PROM), Erasable Programmable Read-Only Memory (EPROM), Electrically Erasable Programmable Read-Only Memory (EEPROM), Flash Memory, other read-only memory, or a combination thereof.

18. The computerized method of claim 16, in which the first computer system comprises a device interface.

19. The computerized method of claim 18, in which the device interface comprises at least one of a keyboard, a keypad, a monitor, a display, a terminal, a computer, a control panel, a vehicle dashboard, a network interface, a machine interface, an electrical interface, an electronic interface, a magnetic interface, an electromagnetic interface including an electromagnetic wave interface, an optical interface, a light interface, an acoustic interface, a video interface, an audio interface, a contactless interface, a mobile phone interface, a smartphone interface, a smartbook interface, a tablet interface, another communication device interface, a Personal Digital Assistant (PDA) interface, a handheld device interface, a portable device interface, a wireless interface, a wired interface, or a combination thereof.

20. The computerized method of claim 16, in which the second computer system comprises a device interface.

21. The computerized method of claim 20, in which the device interface comprises at least one of a keyboard, a keypad, a monitor, a display, a terminal, a computer, a control panel, a vehicle dashboard, a network interface, a machine interface, an electrical interface, an electronic interface, a magnetic interface, an electromagnetic interface including an electromagnetic wave interface, an optical interface, a light interface, an acoustic interface, a video interface, an audio interface, a contactless interface, a mobile phone interface, a smartphone interface, a smartbook interface, a tablet interface, another communication device interface, a Personal Digital Assistant (PDA) interface, a handheld device interface, a portable device interface, a wireless interface, a wired interface, or a combination thereof.
